SEQUENCE LISTING (2 sequences)

SEQ ID NO: 1 - DNA, 22 bp, artificial sequence
  synthetic polynucleotide
cacttggtcatagtcacttgtg

SEQ ID NO: 2 - DNA, 22 bp, artificial sequence
  synthetic polynucleotide
ggatggatgctatgctgcccag

(19) United States
(12) Patent Application Publication
(10) Pub. No.: US 2013/0323761 A1
(43) Pub. Date: Dec. 5, 2013

(54) BIOMARKERS AND THERAPEUTIC TARGETS FOR TREATING CARDIOMYOPATHIES AND CONGESTIVE HEART FAILURE

(71) Applicants: Farah Sheikh, La Jolla, CA (US); Andrew D. McCulloch, San Diego, CA (US); Ju Chen, San Diego, CA (US)

(72) Inventors: Farah Sheikh, La Jolla, CA (US); Andrew D. McCulloch, San Diego, CA (US); Ju Chen, San Diego, CA (US)

(73) Assignee: THE REGENTS OF THE UNIVERSITY OF CALIFORNIA, Oakland, CA (US)

(21) Appl. No.: 13/883,741

(22) PCT Filed: Nov. 9, 2011
(86) PCT No.: PCT/US11/59961

§ 371 Date: Jun. 12, 2013

Related U.S. Application Data
(60) Provisional application No. 61/411,873, filed on Nov. 9, 2010.

(51) Int. Cl. G01N 33/68

(52) U.S. Cl. CPC G01N 33/6887
USPC 435/7.92; 435/15; 435/29; 435/7.1

(57) ABSTRACT

In alternative embodiments, the invention provides methods for predicting or diagnosing a heart disease or a defect in cardiac muscle contractility in an individual, or a defect in rate of cardiac muscle twitch relaxation and/or ventricular torsion, or detecting a cardiac trauma, in an individual or in a cardiac cell, or (by testing) a serum or a blood sample. In alternative embodiments, the invention provides methods for screening for a composition that can treat, ameliorate, prevent or reverse a heart disease or a congestive heart failure in an individual, or a defect in cardiac muscle contractility, or a defect in rate of cardiac muscle twitch relaxation and/or ventricular torsion in an individual or a cardiac muscle cell.

### GOVERNMENT RIGHTS

This invention was made with government support under grant numbers HL096544 and RR008605, both awarded by the National Institutes of Health (NIH). The government has certain rights in the invention.

### TECHNICAL FIELD

This invention generally relates to cell and molecular biology, diagnostics and medicine. In alternative embodiments, the invention provides methods for predicting or diagnosing a heart disease or a defect in cardiac muscle contractility in an individual, or a defect in rate of cardiac muscle twitch relaxation and/or ventricular torsion, or detecting a cardiac trauma, in an individual or in a cardiac cell, or (by testing) a serum or a blood sample. In alternative embodiments, the invention provides methods for screening for a composition that can treat, ameliorate, prevent or reverse a heart disease or a congestive heart failure in an individual, or a defect in cardiac muscle contractility, or a defect in rate of cardiac muscle twitch relaxation and/or ventricular torsion in an individual or a cardiac muscle cell.

### BACKGROUND

Currently, methods for detecting early myocardial dysfunction include the use of cardiac derived biomarkers such as b-type natriuretic peptide, pre-pro-B type natriuretic peptide, and cardiac troponins I and T, and systemically derived markers such as C-reactive protein. These rely on release of inactive proteins into the bloodstream after irreversible cardiac muscle death and an inflammatory response. Although myocardial biopsies may offer unique insights on cardiac disease and failure in select patients, these require complicated invasive procedures that prove to be high risk to the patient/individual. Also, non-invasive imaging modalities are playing an important emerging role in early detection of physical changes to the heart (velocity and displacement as well as strain and strain rate for deformation of muscle) and molecular imaging events in the heart (labeling of metabolites, angiogenic regulators, neuroreceptors, and remodeling factors). However, these molecular events are based on inactive byproducts which are released to the bloodstream, found in the heart as a result of cardiac muscle death, inflammation and/or late-onset diseases processes, and are not necessarily specific to cardiac muscle cells.

### SUMMARY

In alternative embodiments, the invention provides methods for predicting or diagnosing a heart disease or a defect in cardiac muscle contractility in an individual, or a defect in rate of cardiac muscle twitch relaxation and/or ventricular torsion in an individual or in a cardiac cell, or detecting a cardiac trauma, comprising

(a) determining the presence or absence of, or the extent of, myosin light chain-2 (MLC2v) protein phosphorylation in a cardiac cell, an extracellular fluid, a serum or a blood serum or blood sample,

wherein a hypo-phosphorylated MLC2v protein, or non-phosphorylated MLC2v protein in a cardiac cell, and/or release of a phosphorylated MLC2v form into an extracellular fluid, a serum or a blood serum or blood sample, is predictive or diagnostic of a heart disease or a defect in cardiac muscle contractility, or a defect in rate of cardiac muscle twitch relaxation and/or ventricular torsion, or detects a cardiac trauma;

(b) the method of (a), wherein the MLC2v protein is phosphorylated, hypo-phosphorylated or non-phosphorylated in one or more or all serine residues in the MLC2v protein;

(c) the method of (b), wherein the individual, cardiac cell, extracellular fluid, serum or blood serum or sample, is murine or human (murine or human derived);

(d) the method of (c), wherein serine residue 14, or serine residue 15, or serine residue 14 and serine residue 15, or non-human or non-murine equivalent serine residues thereof, are not phosphorylated or phosphorylated;

(e) the method of (a), wherein the MLC2v protein is mutated (is a mutant protein, is a non-wild type protein) in that one or more wild type serine residue or residues is missing or changed to a non-serine amino acid residue or residues;

(f) the method of (e), wherein the MLC2v protein is a mutant protein (non-wild type) such that serine residue 14, or serine residue 15, or serine residue 14 and serine residue 15, or non-human or non-murine equivalent serine residues thereof, are is missing or changed to a non-serine amino acid residue or residues;

(g) the method of (e) or (f), wherein determining whether a MLC2v protein is mutated (is a mutant protein, is a non-wild type protein) is by a method comprising sequencing (all or the relevant part of) the individual's or the cell's genome or transcriptome or MLC2v protein transcript;

(h) the method of any of (a) to (g), wherein the state of hypo-phosphorylation or non-phosphorylation in one or more or all normally (wild type) phosphorylated residues in the MLC2v protein is measured or determined in vitro, ex vivo or in vivo; or

(i) the method of (a), wherein the presence or absence of, or the extent of, myosin light chain-2 (MLC2v) protein phosphorylation in the individual, cardiac cell, extracellular fluid, serum or blood serum or sample, is determined by a method comprising use of an antibody (monoclonal or polyclonal) specific for a phosphorylated serine, or a serine MLC2v S14/S15 phosphorylation site; or comprising use of positron emission tomography (PET), computed tomography (CT), magnetic resonance imaging (MRI), nuclear magnetic resonance imaging (NMRI), or magnetic resonance tomography (MRT) and/or TOI.

In alternative embodiments, the invention provides methods for screening for a composition that can treat, ameliorate, prevent or reverse a heart disease or a congestive heart failure in an individual, or a defect in cardiac muscle contractility, or a defect in rate of cardiac muscle twitch relaxation and/or ventricular torsion in an individual or a cardiac muscle cell, comprising

(1) (a) providing a composition and a cardiac muscle cell, or a cultured cardiac cell, or a cardiac cell extract, or an equivalent cell or extract, or a serum or blood serum or sample;

(b) administering the composition to the cardiac muscle cell, cultured cardiac cell, cardiac cell extract, or equivalent cell or extract, or serum or blood serum or sample; and

(c) measuring or detecting an increase in the relative state of phosphorylation of MLC2v protein in the cardiac muscle cell, cultured cardiac cell, cardiac cell extract, or equivalent cell or extract, or serum or blood serum or sample,

wherein identifying a composition that can increase the relative state of phosphorylation of MLC2v protein in the cardiac muscle cell, cultured cardiac cell, cardiac cell extract, or equivalent cell or extract, or serum or blood serum or sample, identifies a composition that can treat, ameliorate, prevent or reverse a heart disease or a congestive heart failure in an individual, or a defect in cardiac muscle contractility, or a defect in rate of cardiac muscle twitch relaxation and/or ventricular torsion in an individual or a cardiac muscle cell;

(2) the method of (1), wherein the composition comprises a peptide or a protein, a small molecule, a nucleic acid, a carbohydrate or a polysaccharide or a lipid;

(3) the method of (1) or (2), wherein the composition is formulated for administration intravenously (IV), parenterally, orally, or by liposome or vessel-targeted nanoparticle delivery, or the composition comprises a pharmaceutical composition administered in vivo;

(4) the method of any of (1) to (3), wherein the composition increases the activity of or activates a kinase, or a myosin light chain kinase (MLCK); or

(5) the method of (1), wherein the presence or absence of, or the extent of, myosin light chain-2 (MLC2v) protein phosphorylation in a cardiac cell, or serum or blood serum or sample, is determined by a method comprising use of an antibody (monoclonal or polyclonal) specific for a phosphorylated serine, or a serine MLC2v S14/S15 phosphorylation site; or comprising use of positron emission tomography (PET), computed tomography (CT), magnetic resonance imaging (MRI), nuclear magnetic resonance imaging (NMRI), or magnetic resonance tomography (MRT) and/or TOI.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims. Like reference symbols in the various drawings indicate like elements.

FIG. 1: FIGS. 1A and B illustrate and summarize data of two-dimensional gel analysis and mass spectrometry of myofilament proteins analyzing the endogenous regulation of MLC2v phosphorylation in knock-in mice; and FIGS. 1C and D illustrate and summarize myosin light chain kinase (MLCK) phosphorylation assays in Double Mutant (DM) mice: FIG. 1(C) illustrates representative autoradiograms show levels of phosphorylated MLC2v (p-MLC2v) catalyzed by skeletal (top panel) and cardiac (middle panel) MLCK in mice, and FIG. 1(D) graphically summarizes data of phosphorylated MLC2v protein catalyzed by skeletal (top) and cardiac (bottom), as discussed in detail in Example 1, below.

FIG. 2: FIG. 2(A) graphically illustrates a Kaplan-Meier survival curve analysis of mice; FIG. 2(B) graphically illustrates Ventricular weights (VW) to body weight (BW) ratios in WT, SM and DM mice at six months; FIG. 2(C) illustrates representative hearts (top) and sections stained for nuclei and cytoplasm with hematoxylin and eosin, respectively (bottom) from mice at six months; FIG. 2(D) graphically illustrates echocardiographic measurements of WT and DM hearts; FIG. 2(E) graphically illustrates cardiomyocyte length and widths are plotted from WT (black line (n=3)) versus DM (red line) mice at six months; FIGS. 2F, G graphically illustrate data from representative electron micrographs (FIGS. 2F, G) from WT and DM left ventricles at FIG. 2(F) 6 months and FIG. 2(G) 6 weeks; FIG. 2(H) graphically illustrates twitch tension; and FIG. 2(I) graphically illustrates intracellular Ca²⁺ transients, as discussed in detail in Example 1, below.

FIG. 3: FIG. 3(A) schematically illustrates myosin head diffusion (18); FIG. 3(B) schematically illustrates myosin lever arm stiffness; FIG. 3(C) schematically illustrates a computational model of myofilament function; FIG. 3(D) graphically illustrates myofilament model parameters adjusted such that simulations (red line) matched the steady-state force-pCa relation; FIG. 3(E) graphically illustrates Muscle twitch simulations using model parameters of 0% (red trace) and 31% MLC2v-P, as discussed in detail in Example 1, below.

FIG. 4: FIG. 4(A) (top) illustrates urea-glycerol-PAGE, where LV proteins were separated by urea-glycerol-PAGE, transferred to PVDF and stained with Ponceau S (left panel) and blotted with no primary antibody control (lane 1) or MLC2v antibodies (lane 2) (middle panel); a separate gel was stained with phospho-specific Pro-Q Diamond stain (right panel); FIG. 4(A) (middle) illustrates Urea-glycerol-PAGE analysis of MLC2v and MLC2v-P in left ventricular epicardial and endocardial samples from mice; FIG. 4(A) (bottom) illustrates MLC2v-P levels in the LV epicardium and endocardium; FIG. 4(A) also (below PAGE gel illustrations) graphically illustrates data from the PAGE gels, as discussed in detail in Example 1, below.

FIG. 4: FIG. 4(B) graphically illustrates a finite element model of LV function (inset) was driven by MLC2v phosphorylation mechanisms to test the effects of 0% (red trace) and 15% (blue trace) transmural gradients on simulated ventricular torsion over the cardiac cycle; FIG. 4(C) graphically illustrates Ventricular torsion and ejection fraction (EF %) analysis in WT (blue trace) and DM (red trace) hearts using tagged MR imaging (inset, left); FIG. 4(D) illustrates two-dimensional spatial simulations of mechanical work done by muscle fibers across the LV wall during the cardiac cycle in WT and DM hearts, as discussed in detail in Example 1, below.

FIG. 5 (FIG. 5, or FIG. S1) illustrates generation of single (S15A) and double (S14A/S15A) MLC2v phosphorylation mutant knock-in mice; FIG. 5(A) graphically illustrates an MLC2v genomic region of interest (top), the targeting construct (middle), and the mutated S15A locus after homologous recombination (bottom); FIG. 5(B) graphically illustrates an MLC2v genomic region of interest (top), the targeting construct (middle), and the mutated S14A/S15A locus after homologous recombination (bottom); FIG. 5(C, left) illustrates DNAs isolated from Neo-positive SM ES cell clones were digested with SstI and assessed by Southern blotting for wild-type (WT) and heterozygous (HE) alleles with the probe shown in (a); FIG. 5(C, right) illustrates Tail DNAs isolated from WT and SM mice were also analyzed for WT and SM alleles, respectively, by PCR analyses; FIG. 5(D, left) illustrates DNAs isolated from Neo-positive DM ES cell clones were digested with SstI and assessed by Southern blotting for wild-type (WT) and heterozygous (HE) alleles with the probe shown in (b); FIG. 5(D, right) illustrates tail DNAs isolated from WT and DM mice were also analyzed for WT and DM alleles, respectively, by PCR analyses; FIG. 5(E) graphically illustrates incorporation of S15A and S14A/S15A knock-in mutations were verified by PCR and sequencing analyses, as discussed in detail in Example 1, below.

FIG. 6 (FIG. 6, or FIG. S2) graphically illustrates in vivo serial echocardiographic assessment of cardiac size and function in SM mutant versus WT mice, as discussed in detail in Example 1, below.

FIG. 7 (FIG. 7, or FIG. S3) illustrates DCM phenotype in DM mutant mice is not associated with upregulation of cardiac fetal gene molecular marker expression and fibrosis; FIG. 7(A) illustrates a Northern RNA blot showing: atrial natuiretic factor (ANF), α-Myosin Heavy Chain (MHC), β-MHC, cardiac actin (cActin), skeletal α-actin (skActin) and phospholamban (PLB) RNA expression in WT, DM and SM left ventricles; FIG. 7(B) illustrates a Masson Trichrome stain of WT, DM and SM mouse heart sections at three months of age, as discussed in detail in Example 1, below.

FIG. 8 (FIG. 8, or FIG. S4) illustrates a subset of DM mutant mice (DMS) sporadically display cardiac calcification and fibrosis with a modest re-expression of the fetal cardiac marker, β-MHC; FIG. 8A, left, illustrates gross morphology of WT and DMs mouse hearts at three months of age; FIG. 8A, middle left, illustrates cardiac sections from WT and DMS mice were stained with the von Kossa stain; FIG. 8A, right, illustrates a high magnification view of calcification (top, middle) and fibrosis (top, right) in ventricular septum endocardium of DMs mouse heart (DMs-Se); FIG. 8(B) illustrates a Northern RNA blot showing: skActin, β-MHC, α-MHC, ANF RNA expression in representative WT and DMs left ventricles, as discussed in detail in Example 1, below.

FIG. 9 (FIG. 9, or FIG. S5) graphically illustrates echocardiographic measurements of cardiac dimensions and function in WT and DM hearts, as discussed in detail in Example 1, below.

FIG. 10 (FIG. 10, or FIG. S6) graphically illustrates Ca²⁺-contraction twitch dynamics in wild type (WT) and DM muscles, as discussed in detail in Example 1, below.

FIG. 11 (FIG. 11, or FIG. S7) graphically illustrates twitch dynamics in WT and DM muscles; FIG. 11(A) graphically illustrates representative isometric twitch tension measured in right ventricular papillary muscles isolated from WT and DM papillary muscles; FIG. 11(B) graphically illustrates mean characteristics of twitch tension time courses in WT and DM papillary muscles, as discussed in detail in Example 1, below.

FIG. 12 (FIG. 12, or FIG. S8) graphically illustrates DM mutant mice sensitized to pressure overload following transverse aortic constriction (TAC); FIG. 12(A) graphically illustrates left ventricle (LV) to body weight (BW) ratios and in vivo echocardiographic assessment of cardiac size and function in 6 wk old WT and DM mutant mice, before (pre) and following (post) sham and TAC operation for 1 week; FIG. 12(B) graphically illustrates cardiomyocyte length and widths plotted from WT (black line (n=3)) versus DM (red line (n=3)) mice pre and post-sham and TAC operation for 1 week; FIG. 12(C) graphically illustrates ANF, β-MHC, α-MHC, sk-Actin, c-Actin and PLB RNA expression in left ventricles from mice pre and post-sham and TAC operation, as discussed in detail in Example 1, below.

FIG. 13 (FIG. 13, or FIG. S9) illustrates an exemplary schematic model of the mechanisms driving actin-myosin interactions in cardiac muscle, which are controlled by the effects of the myosin accessory protein, MLC2v and its phosphorylation status; FIG. 13 (Top panel) illustrates how MLC2v phosphorylation simultaneously increases the likelihood of myosin binding and force produced by each myosin binding; FIG. 13 (Bottom panel) illustrates loss of these mechanisms results in less actin-myosin binding events, as discussed in detail in Example 1, below.

FIG. 14 (FIG. 14) illustrates data showing how MLC2v protein is detectable in blood serum of mice; and the detection of the phosphorylated form of MLC2v increases following myocardial infarction, as discussed in detail in Example 1, below.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, the present invention provides compositions and methods for early detection of (e.g., predicting) a heart disease and/or heart failure, by identifying and measuring or detecting at least one “active”, early, cardiac-muscle specific biomarker. In one embodiment, the measured or detected biomarkers are predictive of or diagnostic of the ability to maintain normal cardiac function, and when the biomarker is or biomarkers are lost in cardiac cells but released e.g., in serum (e.g., blood serum), this is predictive of or diagnostic of a heart disease and/or a heart failure, e.g., a congestive heart failure, or a cardiac trauma. In alternative embodiments, the invention also provides a therapeutic target that can be used to intervene, e.g., with early defects, leading to heart disease and/or heart failure, e.g., a congestive heart failure, e.g., in cardiac muscle cells and blood serum.

The invention demonstrates a novel biomarker that is predictive of or diagnostic of a heart disease and/or heart failure, e.g., a congestive heart failure, e.g., in cardiac muscle cells. The inventors have identified two phosphorylation sites (S14,S15) on the human cardiac muscle specific gene, ventricular myosin light chain-2 (MLC2v), that makes it “phosphorylation active” and that can (i) be used as an active biomarker to track early disease related events in the heart via its “deactivation” or “de-phosphorylation” and (ii) be used as a therapeutic target to “intervene” with or “re-activate/rescue” the heart, at early stages of disease that lead to congestive heart failure. Thus, in alternative embodiments, the invention provides a therapeutic target for treating, ameliorating, reversing or preventing heart disease and/or heart failure, e.g., a cardiomyopathy and/or a congestive heart failure.

In one embodiment, antibodies specific for MLC2v S14/S15 phosphorylation sites are used to determine the state of phosphorylation in MLC2v protein. In alternative embodiments, sensitive molecular labeled probes (fluorescent, etc.) and imaging agents for detection of MLC2v S14/S15 phosphorylation in the heart are used to detect early events in congestive heart failure. In alternative embodiments, these sensitive molecular labeled probes are used for clinical imaging, diagnostics and prediction of a heart disease using e.g. imaging modalities such as positron emission tomography (PET), computed tomography (CT), magnetic resonance imaging (MRI), nuclear magnetic resonance imaging (NMRI), or magnetic resonance tomography (MRT) and/or TOI.

In alternative embodiments, the invention provides methods for screening for therapeutics, e.g., drugs, which are specific activators (e.g., peptide-based, peptidomimetics, or synthetic drugs) of MLC2v phosphorylation to increase MLC2v S14/S15 phosphorylation in order to reverse early events in a cardiac trauma or a congestive heart failure; and to increase MLC2v phosphorylation, e.g., in cell culture model systems.

In practicing the invention, amplification reactions can be used to quantify the presence and/or amount of nucleic acid in a sample (e.g., whether a MLC2v gene or transcript is a wild type or variant, e.g., variant allele), to label a nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; and Sooknanan (1995) Biotechnology 13:563-564.

In practicing the invention, any protocol known in the art can be used to detect phosphorylation, or the extent of phosphorylation, of a protein (e.g., a MLC2v protein), including e.g., antibodies that only detect phosphorylated forms of a protein or the protein (e.g., a MLC2v), one and two dimensional gels (e.g., SDS-PAGE), chromatography, quantitative protein phosphorylation methods such as fluorescence immunoassays (e.g., using a dinuclear metal-chelate phosphate recognition unit and a sensitive fluorophore), Microscale Thermophoresis, Förster resonance energy transfer (FRET), time-resolved fluorescence (TRF), fluorescence polarization, fluorescence-quenching, mobility shift, bead-based detection, in situ proximity ligation assays (e.g., DUOLINK®, Olink Bioscience), and cell-based formats, and the like. For example, in some embodiments, Mass Spectrometry (MS) or LC-MS methods can be used to quantify gel-separated proteins and their sites of phosphorylation, e.g., as described by Cutillas (2005) Molecular & Cellular Proteomics 4:1038-1051. In other embodiments, an automated LC/MS/MS approach is used, e.g., as described by Williamson (2006) Mol. Cell. Proteomics 5:337-346, describing use of a Hybrid Triple Quadrupole Linear Ion Trap Mass Spectrometer.

In alternative embodiments, mass spectrometric techniques such as collision-induced dissociation (CID) and electron transfer dissociation (ETD) are used, e.g., to provide a comprehensive parallel analysis of peptide sequences and phosphorylation.

In one embodiment, a Western blot, the most common method used for assessing the phosphorylation state of a protein, is used: e.g., following separation of the biological sample with SDS-PAGE and subsequent transfer to a membrane (usually PVDF or nitrocellulose), a phospho-specific antibody can be used to identify the protein of interest.

In one embodiment, an ELISA is used. It has become a powerful method for measuring protein phosphorylation. ELISAs can be more quantitative than Western blotting and show great utility in studies that modulate kinase activity and function. The format for this microplate-based assay typically utilizes a capture antibody specific for the desired protein, independent of the phosphorylation state. The target protein, either purified or as a component in a complex heterogeneous sample such as a cell lysate, is then bound to the antibody-coated plate. A detection antibody specific for the phosphorylation site to be analyzed is then added. These assays are typically designed using colorimetric or fluorometric detection. The intensity of the resulting signal is directly proportional to the concentration of phosphorylated protein present in the original sample.

In one embodiment, protein phosphorylation within intact cells is determined; this protocol can be more accurate in representing phosphorylation status; and any one of several immunoassays enabling the measurement of protein phosphorylation in the context of a whole cell can be used. For example, the cells can be fixed and blocked in the same well. Phospho-specific antibodies can be used to assess phosphorylation status using fluorometric or colorimetric detection systems. These assays can bypass the need for the creation of cell lysates; and can be used in high throughput analyses.

In one embodiment, protein phosphorylation is determined using intracellular flow cytometry and immunocytochemistry/immunohistochemistry (ICC/IHC); for example, flow cytometry can be used with a laser to excite a fluorochrome for antibody detection; filter sets and fluorochromes with non-overlapping spectra can be used for assessing multiple proteins in the same cell. Flow cytometry can be used in rapid, quantitative, single cell analyses.

In some embodiments, e.g., when using MS, enrichment strategies for phospho-protein analysis can be used, e.g., including immobilized metal affinity chromatography (IMAC), phosphospecific antibody enrichment, chemical-modification-based methods such as beta-elimination of phospho-serine and -threonine, and replacement of the phosphate group with biotinylated moieties.

### Kits

The invention provides kits comprising compositions used to practice methods of this invention, e.g. optionally including instructions for practicing and interpreting results of practicing methods of the invention, or any combination thereof. As such, kits comprising PCR primers, probes, antibodies, cells, vectors and the like are provided herein.

The invention will be further described with reference to the following examples; however, it is to be understood that the invention is not limited to such examples.

### EXAMPLES

### Example 1

### Methods of the Invention are Effective for Predicting or Diagnosing, or Treating or Preventing, Heart Disease

The data presented herein demonstrates methods of the invention are effective for predicting or treating, ameliorating, reversing or preventing heart disease and/or heart failure, e.g., a cardiomyopathy and/or a congestive heart failure.

The inventors provide evidence of the existence of a myosin light chain-2 phosphorylation gradient in the heart in vivo and demonstrate that specific MLC2v phosphorylation sites (S14/S15) are important in the pathogenesis of congestive heart failure and that MLC2v is detectable in blood serum and that the phospho-specific form of MLC2v is increased in blood serum following cardiac injury or trauma, e.g., such as after a myocardial infarction or related injury. The inventors have shown that loss in MLC2v S14/S15 phosphorylation and its mechanisms in the mouse heart in vivo, predicts dilated cardiomyopathy and congestive heart failure even before classic early makers, such as ultrastructural sarcomeric defects and molecular markers (e.g., ANF, BNP, skeletal alpha-actin, etc.) associated with cardiac stress.

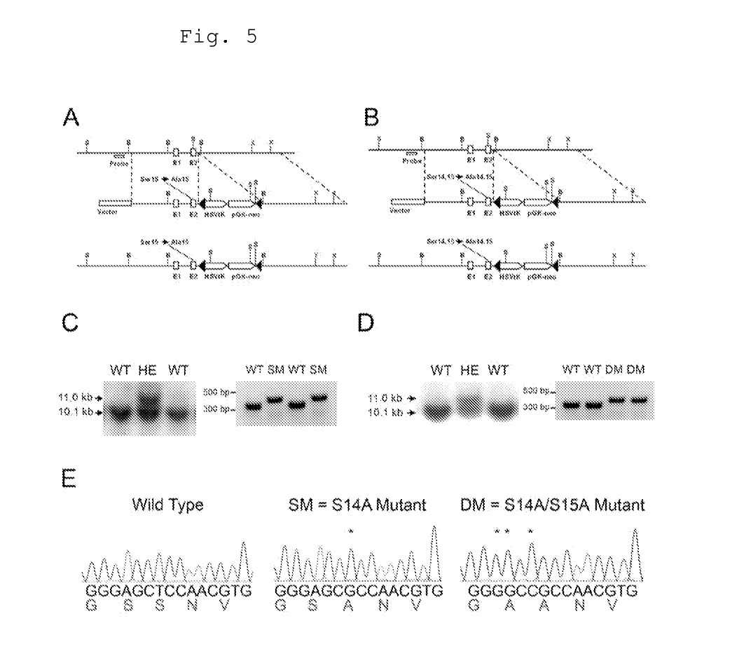

To determine the functional relevance of the regulation of myosin accessory protein, MLC2v via its phosphorylation in the heart in vivo, we generated two novel MLC2v phosphorylation mutant mouse lines (FIG. S1, or FIG. 5). Although the endogenous regulation of MLC2v phosphorylation in vivo remains unclear, it is thought that Serine 15 (S15) on MLC2v is the major phosphorylation site regulating muscle function in rodents and humans (6,10-12, 13-15), whereas Serine 14 (S14) is also thought to be important for stretch-activation responses in muscle (14). To determine the role(s) of MLC2v S15 and S14 phosphorylation sites in vivo, we generated knock-in mouse mutants targeting Ser15Ala (S15A) and Ser14Ala(S14A)/Ser15Ala(S15A) (FIG. S1, or FIG. 5). Incorporation of the mutations in both S15A (Single Mutant, SM) and S14A/S15A (Double Mutant, DM) mice was confirmed using DNA analyses (FIG. S1, or FIG. 5).

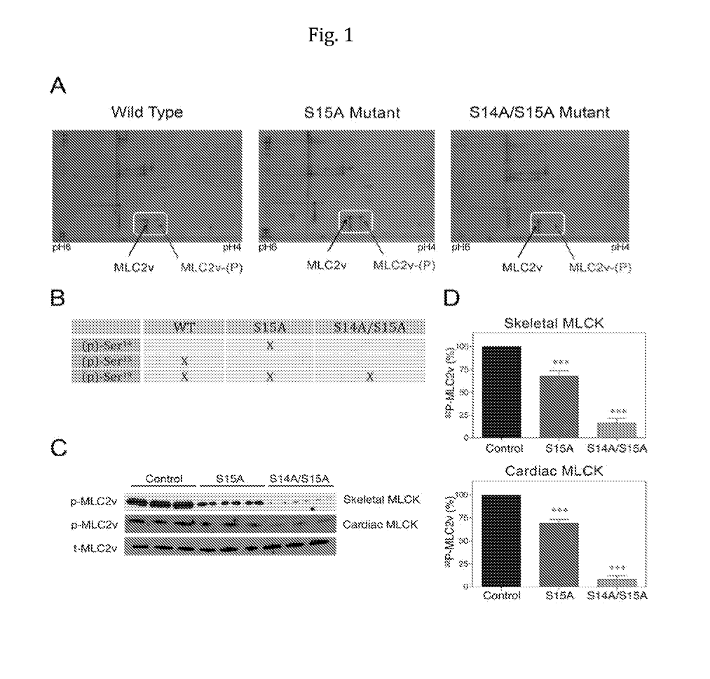

The endogenous regulation of MLC2v phosphorylation in our knock-in mice was analyzed using two-dimensional gel analysis and mass spectrometry of myofilament proteins (FIGS. 1A, B). WT hearts revealed an approximately 31% of MLC2v phosphorylation (FIG. 1A) corresponded to endogenous phosphorylation at S15 and S19 sites (FIG. 1B). Surprisingly, SM mutant hearts displayed a compensatory increase (69% of total MLC2v) in MLC2v phosphorylation (FIG. 1A), which corresponded to an endogenous switch to S14 phosphorylation in vivo (FIG. 1B). Loss in MLC2v phosphorylation was only seen in DM myocardium where there was loss of S14 and S15 phosphorylation (FIGS. 1A, B). These results suggest that the contribution of S19, important for smooth muscle MLC2 phosphorylation (16), is likely negligible in regulating endogenous MLC2v phosphorylation in DM cardiac muscle. Myosin light chain kinase (MLCK) phosphorylation assays (FIGS. 1C, D) also revealed that a significant decrease in MLC2v phosphorylation was observed in DM mice. These results altogether indicate that S14 and S15 are both necessary and sufficient to significantly reduce endogenous MLC2v phosphorylation in vivo.

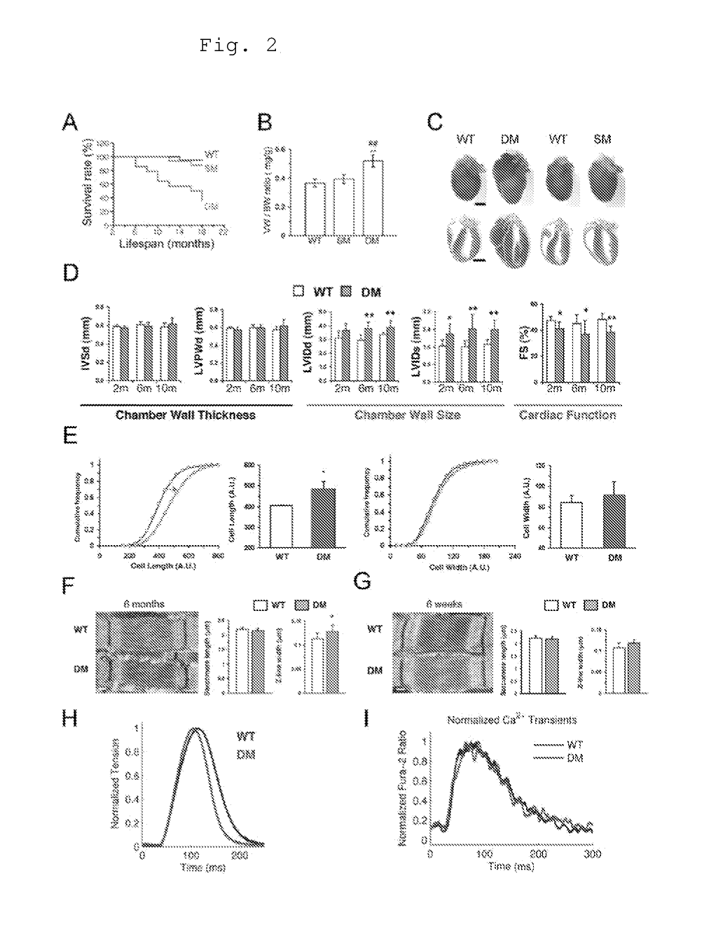

DM mutant mice are viable at birth; however, they display a striking susceptibility to premature death (FIG. 2A) due to heart failure as a consequence of dilated cardiomyopathy (DCM) (FIG. 2B-E). Features of DM hearts included significant increases in (i) ventricular weight to body weight ratios (FIG. 2B), (ii) age-dependent cardiac chamber enlargement, which was accompanied by a significant decrease in cardiac function (FIG. 2C-D), (iii) cardiomyocyte cell length but not width changes (FIG. 2E) as well as (iv) classical ultrastructural sarcomeric defects, which included significant Z-line thickening at six months of age (FIG. 2F).

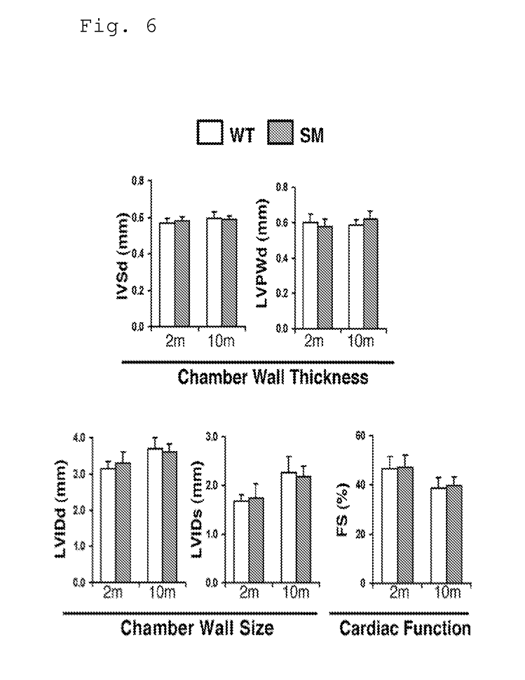

The effects on heart structure, function and premature death in DM mice were not observed in SM mice (FIG. S2, or FIG. 6), suggesting that the endogenous switch to S14 phosphorylation (FIGS. 1A, B) is sufficient to functionally compensate for the loss of MLC2v S15 in vivo.

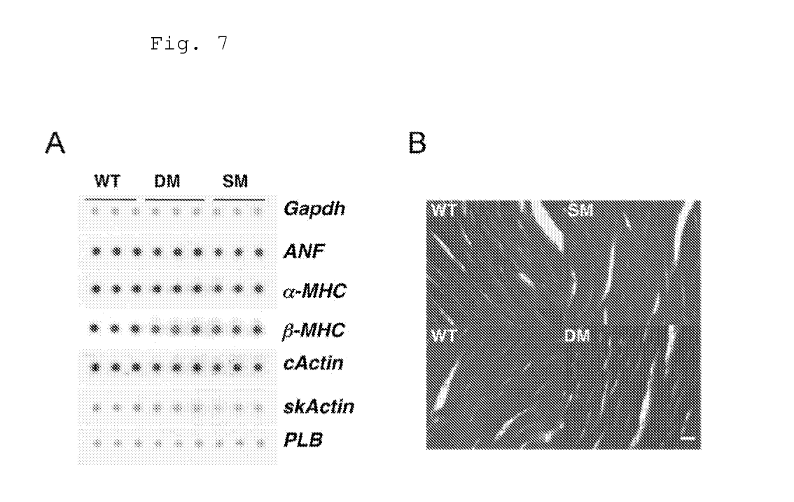

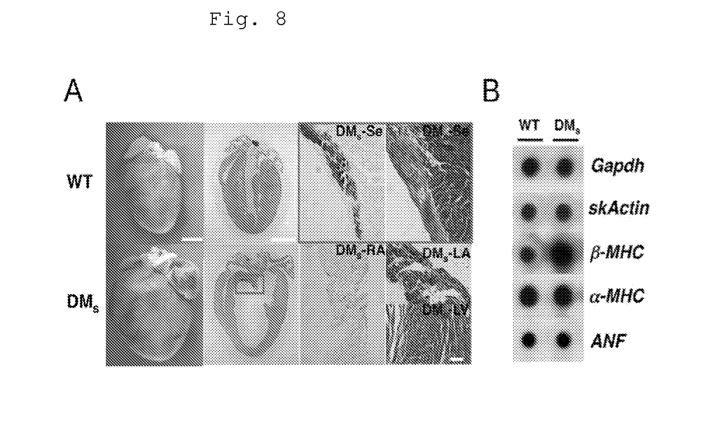

Most remarkably, the heart muscle defects observed in DM mutant mice were not associated with significant changes in the cardiac expression of fetal genes, which are classically associated with early signs of cardiac stress (FIG. S3A, or FIG. 7A). In addition, the majority of DM mice did not exhibit signs of heart muscle damage or fibrosis when compared to WT and SM (FIG. S3B, or FIG. 7B). However, in sporadic cases (10%), DM mutant mice displayed heterogenous calcification and fibrosis most prominently in the ventricular endocardium associated with low-level expression of a single fetal gene, beta-MHC (β-MHC) (FIG. S4, or FIG. 8), highlighting a more severe disease consequence for loss of MLC2v phosphorylation in the sub-endocardial layer of the heart. These results highlight a direct influence of loss of MLC2v phosphorylation on heart disease and failure in vivo, which now proposes and resolves its direct contribution to human cardiomyopathy and failure (6-12). We further outline the phosphorylation sites that are important for ML2v phosphorylation regulation in disease in vivo, which was also previously unclear in rodents and humans (6-12,15).

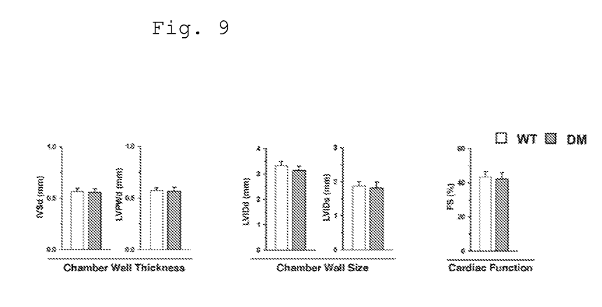

To determine the primary functional consequences of an absence of MLC2v phosphorylation in intact cardiac muscle that could account for the end-stage heart disease and failure in DM mutant mice, we simultaneously measured twitch tension and Ca²⁺ transients from WT and DM mutant muscles at 6 wks of age. At this age, DM mutant mice did not exhibit defects in myocardial ultrastructure, dimensions and global cardiac function when compared to WT mice (FIG. 2G; FIG. S5, or FIG. 9).

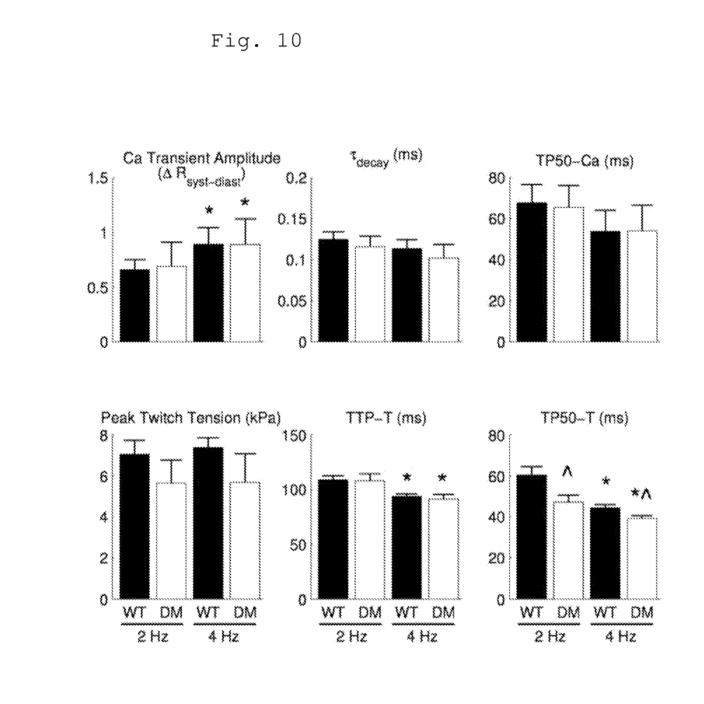

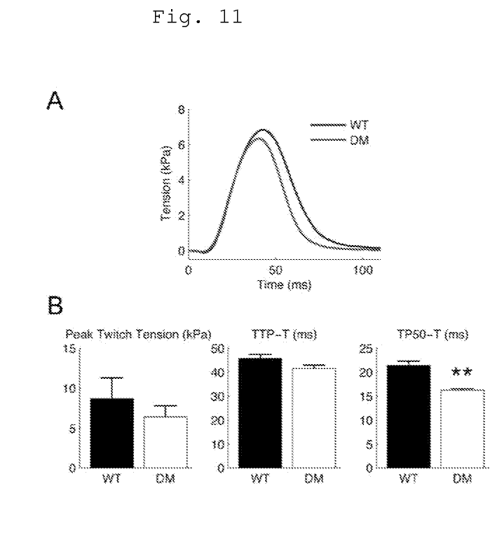

We demonstrate that DM mutant cardiac muscle only exhibited significant differences in the timing of twitch tension that resulted in faster twitch relaxation than WT muscles (FIG. 2H; FIG. S6, or FIG. 10). These same specific twitch contraction defects were observed at 37° C., indicating that they are also present in DM mutant mice under physiological conditions (FIG. S7, or FIG. 11).

Moreover, the defects in the rate of twitch relaxation in DM mutant muscles were also observed in the absence of calcium cycling defects as evidenced by the similarly observed robust Ca²⁺ transients in WT and DM mutant cardiac muscles (FIG. 2I). These results suggest a specific direct role for MLC2v phosphorylation in cardiac muscle relaxation kinetics.

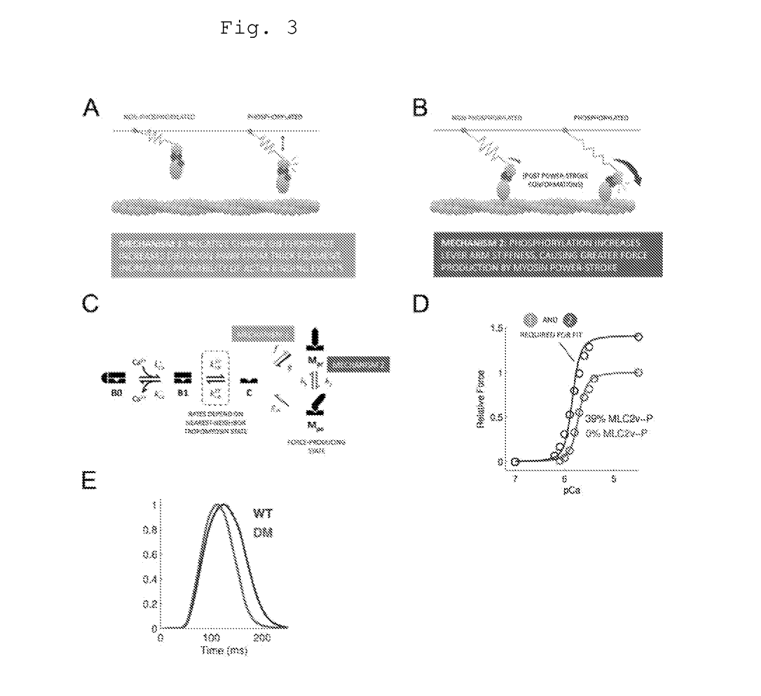

To define the precise molecular mechanisms underlying a role for MLC2v phosphorylation in cardiac muscle, we created a new computational model of myofilament Ca²⁺ activation based on our recent work (17), which tested two novel mechanisms for a direct role for MLC2v phosphorylation in the kinetics of actin-myosin regulation of cardiac muscle (FIG. 3A-C). Since the precise mechanisms underlying a role for MLC2v phosphorylation in cardiac muscle are unknown, we exploited information gained from in vitro studies in skeletal muscle, which suggested that the negative charge from phosphorylation could increase the diffusion of myosin heads away from the thick filament backbone and toward actin (FIG. 3A; Mechanism 1) (18). Recent work with isolated skeletal myosins also independently suggested that MLC2 phosphorylation could increase the stiffness of the myosin lever arm (FIG. 3B; Mechanism 2) (19). The global effect of MLC2v phosphorylation via the MLC kinase is an increase in the contractile force of skinned cardiac myofilaments as well as its sensitivity to Ca²⁺ in vitro (20, 21). As a result, we used this functional observation as a readout to test the computational fit of either of these hypothesized mechanisms (or in combination) to quantitatively match these in vitro observations in cardiac muscle as reported by Stelzer et al. (FIG. 3C) (21). We show with simulations that both Mechanism 1 and 2 are necessary and sufficient in equal proportions to match all essential characteristics of the published data observed in skinned cardiac myofilaments in vitro (FIG. 3D, Tables S1 and S2) (20,21). Neither mechanism on its own nor permutations that were disproportionate from each other were capable of matching the reported effects of MLC2v phosphorylation in cardiac muscle (data not shown). Our observations could only be achieved when slowing of the cross-bridge power stroke step due to elevated stiffness of the myosin lever arm or neck domain (Mechanism 2) counterbalanced the increase in myosin binding (Mechanism 1), causing an increased accumulation of crossbridges in the pre-power stroke, non-force generating state (Mₚᵣ, FIG. 3C). This population of attached cardiac muscle myosin heads then increases myofilament Ca²⁺ sensitivity by cooperatively recruiting neighbouring myosin binding sites, while being poised to increase the likelihood of force produced by each myosin binding event (FIG. 3C). These results define a novel indispensable functional role for MLC2v phosphorylation in directly controlling actin-myosin interactions in cardiac muscle through regulation of myosin turnover kinetics, while revealing how these two molecular mechanisms act in concert to explain global effects of MLC2v phosphorylation on cardiac myofilament contractility in vitro (20, 21). We further demonstrate that our computational model could predict twitches in the absence and presence of MLC2v phosphorylation (FIG. 3E) that matched twitch measurements in intact WT and DM mutant cardiac muscles, respectively (FIG. 2H; FIG. S7, or FIG. 11).

Thus, we also resolve the direct phosphorylation-dependent mechanism defects that account for the accelerated twitch relaxation (FIGS. 2H, 3E) observed in DM muscles. Our studies also identify previously unrecognized defects in rate of twitch relaxation, which precede heart failure before the appearance of classic early ultrastructural (FIG. 2G) and calcium cycling (FIG. 2I) alterations associated with heart disease.

These studies further provide new insights on direct mechanisms controlling rate of cardiac muscle twitch relaxation, which function independent of actin-bound regulatory proteins and rely on myosin kinetics.

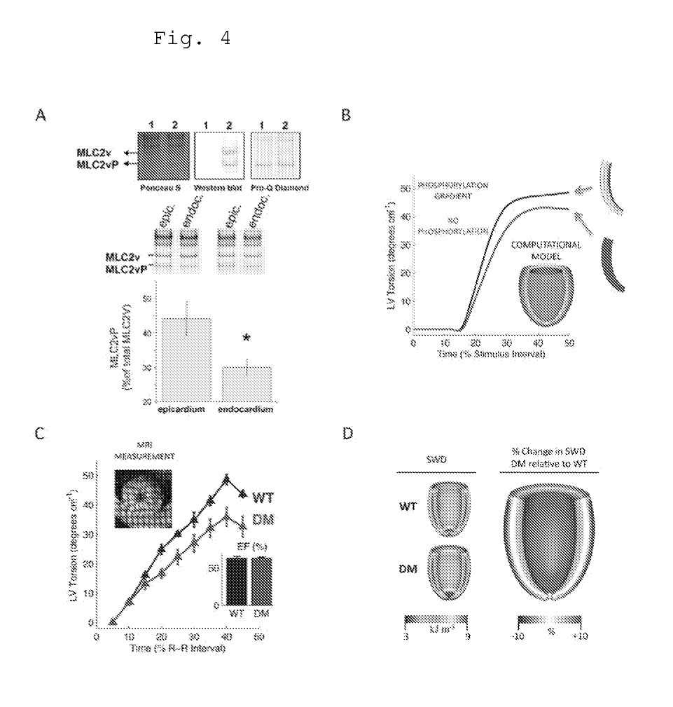

To determine how these primary defects in myosin kinetics resulting from loss of MLC2v phosphorylation could affect the heart in vivo, we first assessed the endogenous expression pattern of MLC2v phosphorylation in the mouse heart. We show that MLC2v phosphorylation is heterogenous and exists as a transmural gradient in the mouse LV wall (FIG. 4A), decreasing from epicardium to endocardium (44.4±9.6% versus 30.2±4.5%, p<0.03), which is consistent with previous findings in rodent and human hearts (6, 22). Since it is thought that these gradients drive the twisting motion of the heart (torsion) (6), we created finite element models of LV mechanics driven by either WT (phosphorylation gradient) or DM (no phosphorylation gradient) twitch characteristics (FIG. 4B) to predict direct contributions of this non-uniform MLC2v phosphorylation gradient on LV torsion. Simulations representing the absence of MLC2v phosphorylation exhibited strikingly lower values of LV torsion (twist) throughout systole (FIG. 4B). To determine whether these model predictions also translate in vivo, the time course of LV torsion was measured in WT and DM mutant mice at 6 wks of age, during the systolic interval (FIG. 4C) using phase-tagged magnetic resonance (MR) imaging. Torsion time course between WT and DM mutant hearts were also significantly different (p<0.05). Maximum torsion values for WT and DM mutant mice at 6-wks of age were 49±2 and 36±5 degrees cm⁻¹, respectively, and differed significantly (p<0.05). No significant differences in global cardiac function as measured by % ejection fraction (EF) via MR were observed between WT and DM mutant hearts at this stage (FIG. 4C), suggesting that the early torsion defects were not due to overt global effects on the heart.

Replacement of the computational transmural gradient with a uniform MLC2v phosphorylation across the heart did not maintain torsion to the extent experimentally observed in WT hearts in vivo (data not shown), further validating the importance of these gradients in normal cardiac function. These observations also reveal novel unrecognized pre-failure consequences of loss of myosin turnover kinetics in vivo that result in decreased ventricular torsion. The mechanisms underlying torsion are fundamental to understand since decreased ventricular torsion is emerging as an early clinical predictor of heart disease in children and adults (23, 24) and it is also severely depressed in patients with DCM (25); however, the mechanisms underlying these events are currently unknown. Thus, our novel mouse model can also be exploited to further understand the mechanisms underlying LV torsion in humans; since torsion was shown to be physiologically equivalent in mice and man (26).

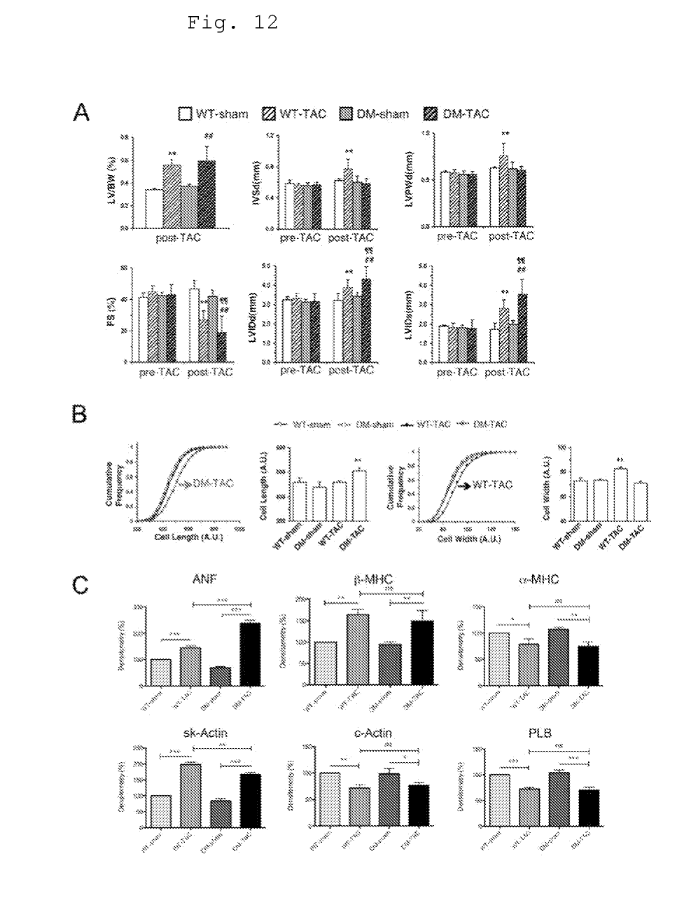

To determine how an underlying loss in LV torsion in the absence of overt ultrastructural sarcomeric defects could account for the DCM and heart failure in DM mutant mice, we exploited our finite element model of LV mechanics to determine the consequences of loss of MLC2v phosphorylation-dependent mechanisms on myofiber strain kinetics across the heart wall (epicardium to endocardium), as measured by myofiber stroke work density (SWD; FIG. 4D). Model predictions demonstrate that to maintain EF under conditions of reduced levels of LV torsion, the DM mutant heart would undergo ˜10% increase in subendocardial myofiber SWD above WT hearts (FIG. 4D). This reveals that loss of phosphorylation redistributes stress in myofibers across the heart wall, with direct and clear adverse consequences on the subendocardial workload (FIG. 4D). Since the subendocardial myofibers are known to be particularly vulnerable in patients with DCM and the mechanisms are unknown (27), our studies offer new insights of how defects in transmural-based MLC2v phosphorylation-mediated mechanisms contribute to decreased subendocardial workload and DCM. The consequence of this defective subendocardial workload is further highlighted by the previously observed increase in disease vulnerability of the ventricular subendocardium in a subset of DM mutant mice exhibiting DCM (FIG. S4, or FIG. 8) and also by the increased vulnerability of DM mice to DCM following induction of mechanical (LV pressure overload) stress at an early stage when no structural/disease alterations are observed (FIG. S8, or FIG. 12). Pressure overload induced different myocardial growth responses in DM mutant compared to WT mice undergoing similar molecular and trans-stenotic pressure gradient stresses (FIG. S8, or FIG. 12), in the sense that DM hearts exhibited only increases in chamber size and not chamber wall thickness, indicative of DCM, as opposed to expected increases in both chamber size and wall thickness observed in WT hearts, which typically undergo concentric hypertrophy (FIG. S8, or FIG. 12A). Cardiomyocytes from DM mice exhibited these same growth response defects following pressure overload in that cell length was only increased in DM mice, while cell width was expectedly increased in WT mice (FIG. S8, or FIG. 12B). These results further reveal that S15 and S14 phosphorylation sites on MLC2v (cardiomyocyte cytoskeleton) can be uncoupled from cardiac stress-related transcriptional machinery (molecular markers) and also act as critical signaling effectors for strains/stress, which are important in controlling cardiac muscle cell growth responses within the heart.

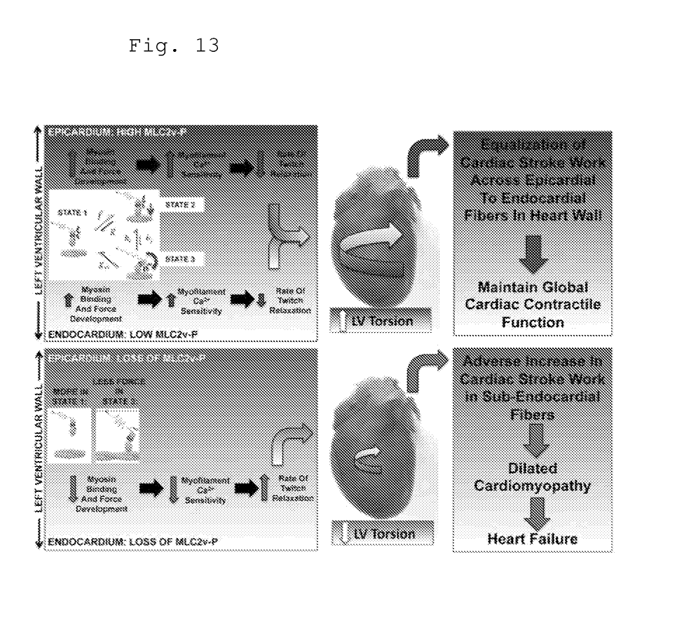

Using integrative gene-targeted animal and computational approaches, we provide compelling evidence of an indispensable and direct influential role for myosin accessory proteins, like MLC2v, in controlling actin-myosin interactions independent of actin-bound regulatory proteins, which when lost have a direct impact on heart disease and failure (FIG. S9, or FIG. 13). Our studies also uncover how defects in these unrecognized underlying mechanisms track and anticipate early events in heart disease and failure. Our data have a potential impact on our understanding of (i) ventricular function, by evolving current perceptions and resolving a role for myosin accessory proteins in actin-myosin regulation in cardiac muscle (1), (ii) a previously unappreciated role for myosin-based phosphorylation gradients to preserve normal cardiac function via LV torsion, which could allow us to overcome current challenges for long-term successful clinical outcomes of using stem cell and tissue engineering approaches as therapies for cardiac muscle dysfunction and disease (reviewed in 28), and (iii) alternative applications of these findings; thus, alternative embodiments provide protein-based assays to detect heart defects, which include sensitive image-based methods to analyze epicardial MLC2v phosphorylation to mark early events in heart disease.

In alternative embodiments, methods of the invention are used in multi-scale computational models and image-based approaches for the diagnosis, prevention, and improved management of direct and early events in human heart disease.

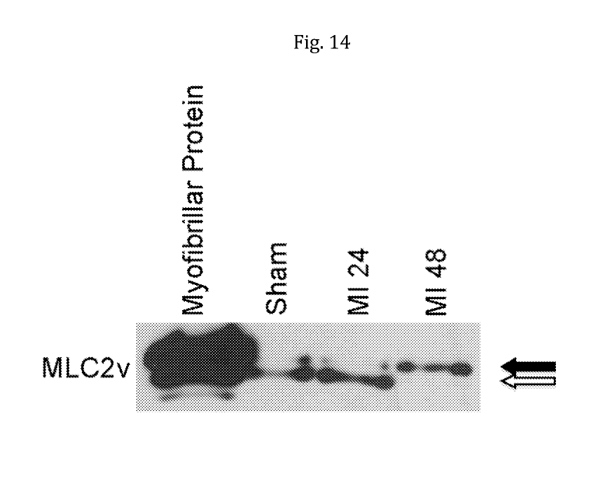

We also show that MLC2v is detectable in blood serum and that the phospho-specific form of MLC2v is increased in blood following cardiac injury, in this embodiment, a myocardial infarction, reinforcing that MLC2v phosphorylation is an important biomarker to detect in blood serum, which further highlights its mechanistic relevance to the pathogenesis of heart failure, as illustrated in FIG. 14.

### FIGURE LEGENDS

FIG. 1. Endogenous MLC2v phosphorylation in the novel MLC2v mutant mouse lines in vivo.
FIG. 1(A) illustrates a Two-dimensional gel analysis of MLC2v in myofilament proteins in mice at 6 wks of age. Silver stained gels were used to determine percentage of MLC2v phosphorylation by densitometry as shown in the representative gels. Unphosphorylated (left) and phosphorylated (right) MLC2v are highlighted in gels.
FIG. 1(B) is a summary table depicting the mass spectrometry analysis of endogenous MLC2v Ser-14, Set-15 and Ser-19 phosphorylation in myofilament proteins in mice at 6 wks of age. Raw data and search result files for mass spectrophometry analysis on mice is available as supplementary material if required.
FIG. 1(C) illustrates representative autoradiograms show levels of phosphorylated MLC2v (p-MLC2v) catalyzed by skeletal (top panel) and cardiac (middle panel) MLCK in mice (n=3). Total MLC2v (t-MLC2v) is shown as a loading control.
FIG. 1(D) graphically summarizes data of phosphorylated MLC2v protein catalyzed by skeletal (top) and cardiac (bottom); MLCK was quantified through liquid scintillation counting in SM and DM mice and expressed as a percentage of WT, which are set to 100%. Percentage values are expressed as mean±SEM (n=3). ***p<0.001.
FIG. 2. DM mutant display premature death due to heart failure in the form of dilated cardiomyopathy and early defects in twitch relaxation.
FIG. 2(A) graphically illustrates a Kaplan-Meier survival curve analysis of mice.
FIG. 2(B) graphically illustrates Ventricular weights (VW) to body weight (BW) ratios in WT (n=7), SM (n=10) and DM (n=5) mice at six months. **p<0.01 DM versus WT mice, ## p<0.01 DM versus SM mice.
FIG. 2(C) illustrates representative hearts (top) and sections stained for nuclei and cytoplasm with hematoxylin and eosin, respectively (bottom) from mice at six months. Bar=2 mm.
FIG. 2(D) graphically illustrates echocardiographic measurements of WT (n=6 at two months; n=10 at six months; n=10 at ten months) and DM (n=6 at two months; n=9 at six months; n=8 at ten months) hearts. IVSd: Interventricular septal wall thickness at end-diastole; LVPWd: Left ventricular (LV) posterior wall thickness at end-diastole; LVIDd: LV internal dimension at end-diastole; LVIDs: LV internal dimension at end-systole; FS (%): LV percent fraction shortening. *p<0.05, **p<0.01
FIG. 2(E) graphically illustrates cardiomyocyte length and widths are plotted from WT (black line (n=3)) versus DM (red line (n=3)) mice at six months. Red arrow highlights shift towards higher cell length.

They are expressed as arbitrary units (A.U.). *p<0.05; FIGS. 2F, G graphically illustrate data from representative electron micrographs (FIGS. 2F, G) from WT and DM left ventricles at FIG. 2(F) 6 months and FIG. 2(G) 6 weeks. Sarcomere length and Z line widths (n=>100 per heart, n=3) was determined Bar=200 nm. *p<0.05.
FIG. 2(H) graphically illustrates twitch tension; and FIG. 2(I) graphically illustrates intracellular Ca²⁺ transients, which were measured in 6 wk old papillary muscles at 25° C. Leftward shift highlights accelerated twitch relaxation.
FIG. 3 illustrates a novel computational model of the invention that identifies a dual molecular role for ventricular myosin light chain phosphorylation (MLC2v-P) in regulation of actin-myosin interactions in cardiac muscle that also underlies the twitch relaxation defects in DM mice. Two mechanisms tested include the effects of MLC2v-P on:
FIG. 3(A) schematically illustrates myosin head diffusion (18) and FIG. 3(B) schematically illustrates myosin lever arm stiffness (19). FIG. 3(C) schematically illustrates a computational model of myofilament function (17) that includes a three-state cross bridge cycle, allowing for novel quantitative representation of both MLC2v-P mechanisms (orange, Mechanism 1; green, Mechanism 2). Model parameters are described in Methods and Table S1.
FIG. 3(D) graphically illustrates myofilament model parameters were adjusted such that simulations (red line) matched the steady-state force-pCa relation reported in dephosphorylated skinned mouse myocardium measurements (data points digitized from Ref 21 red open circles) representing conditions of 0% MLC2v-P. A fit was obtained only when both mechanisms were represented in equal proportions (Table S2).
FIG. 3(E) graphically illustrates muscle twitch simulations using model parameters of 0% (red trace) and 31% MLC2v-P (blue trace, corresponding to measured endogenous MLC2v-P levels in WT myocardium in FIG. 1A). Similar defects in muscle twitch shape, representing accelerated twitch relaxation, were observed in 0% MLC2v-P simulations as in intact DM muscles.
FIG. 4 illustrates MLC2v phosphorylation mediated-mechanisms of the invention that underlie the pre-failure defects in ventricular torsion and subendocardial workload in DM hearts in vivo.
FIG. 4(A) (top) illustrates urea-glycerol-PAGE, where LV proteins were separated by urea-glycerol-PAGE, transferred to PVDF and stained with Ponceau S (left panel) and blotted with no primary antibody control (lane 1) or MLC2v antibodies (lane 2) (middle panel). A separate gel was stained with phospho-specific Pro-Q Diamond stain (right panel). Combined methods identified MLC2v and MLC2v-P bands. FIG. 4(A) (middle) illustrates Urea-glycerol-PAGE analysis of MLC2v and MLC2v-P in left ventricular epicardial and endocardial samples from mice. FIG. 4(A) (bottom) illustrates MLC2v-P levels in the LV epicardium and endocardium (0 mmHg) is based on loading a range of volumes from the same solubilized sample on urea glycerol PAGE. Integrated optical density method was used to determine MLC2v-P level as a percentage of MLC2v. FIG. 4(A) also (below PAGE gel illustrations) graphically illustrates data from the PAGE gels, expressed as mean±SEM (n=4). *p<0.05.
FIG. 4(B) graphically illustrates a finite element model of LV function (inset) was driven by MLC2v phosphorylation mechanisms to test the effects of 0% (red trace) and 15% (blue trace) transmural gradients on simulated ventricular torsion over the cardiac cycle (% Stimulus Interval).
FIG. 4(C) graphically illustrates Ventricular torsion and ejection fraction (EF %) analysis in WT (blue trace) and DM (red trace) hearts using tagged MR imaging (inset, left). % EF was not changed between WT (blue bar) and DM (red bar) hearts (inset, right). Values are expressed as mean±SEM (n=3).
FIG. 4(D) illustrates two-dimensional spatial simulations of mechanical work done by muscle fibers across the LV wall during the cardiac cycle (cardiac stroke work density, SWD) in WT and DM hearts. Percent change in SWD in DM relative to WT hearts is shown.
FIG. 5 (or FIG. S1) illustrates generation of single (S15A) and double (S14A/S15A) MLC2v phosphorylation mutant knock-in mice.
FIG. 5(A) graphically illustrates an MLC2v genomic region of interest (top), the targeting construct (middle), and the mutated S15A locus after homologous recombination (bottom).
FIG. 5(B) graphically illustrates an MLC2v genomic region of interest (top), the targeting construct (middle), and the mutated S14A/S15A locus after homologous recombination (bottom).
FIG. 5(C, left) illustrates DNAs isolated from Neo-positive SM ES cell clones were digested with SstI and assessed by Southern blotting for wild-type (WT) and heterozygous (HE) alleles with the probe shown in (a).
FIG. 5(C, right) illustrates Tail DNAs isolated from WT and SM mice were also analyzed for WT and SM alleles, respectively, by PCR analyses.
FIG. 5(D, left) illustrates DNAs isolated from Neo-positive DM ES cell clones were digested with SstI and assessed by Southern blotting for wild-type (WT) and heterozygous (HE) alleles with the probe shown in (b).
FIG. 5(D, right) illustrates tail DNAs isolated from WT and DM mice were also analyzed for WT and DM alleles, respectively, by PCR analyses.
FIG. 5(E) graphically illustrates incorporation of S15A and S14A/S15A knock-in mutations were verified by PCR and sequencing analyses. Mutations are highlighted by asterisks (*).
FIG. 6 (or FIG. S2) graphically illustrates in vivo serial echocardiographic assessment of cardiac size and function in SM mutant versus WT mice at two (WT n=9; SM n=12) and ten (WT n=9; SM n=12) months of age. Abbreviations: IVSd: Interventricular septal wall thickness at end-diastole; LVPWd: Left ventricular (LV) posterior wall thickness at end-diastole; LVIDd: LV internal dimension at end-diastole; LVIDs: LV internal dimension at end-systole; FS (%): LV percent fraction shortening.
FIG. 7 (or FIG. S3) illustrates DCM phenotype in DM mutant mice is not associated with upregulation of cardiac fetal gene molecular marker expression and fibrosis.
FIG. 7(A) illustrates a Northern RNA blot showing: atrial natuiretic factor (ANF), α-Myosin Heavy Chain (MHC), β-MHC, cardiac actin (cActin), skeletal α-actin (skActin) and phospholamban (PLB) RNA expression in WT, DM and SM left ventricles (n=3) at three months of age. Gapdh RNA was assessed as a loading control. Similar results were obtained in mice at 6 weeks of age (data not shown).
FIG. 7(B) illustrates a Masson Trichrome stain of WT, DM and SM mouse heart sections at three months of age. Bar is equivalent to 50 μm.
FIG. 8 (or FIG. S4) illustrates a subset of DM mutant mice (DMS) sporadically display cardiac calcification and fibrosis with a modest re-expression of the fetal cardiac marker, β-MHC.
FIG. 8(A, left) (left) illustrates gross morphology of WT and DMs mouse hearts at three months of age. Bar is equivalent to 2 mm. FIG. 8A, middle left, illustrates cardiac sections from WT and DMS mice were stained with the von Kossa stain. Bar is equivalent to 2 mm. Red square highlights calcification in ventricular septum endocardium of DMs mouse heart. FIG. 8A, right, illustrates a high magnification view of calcification (top, middle) and fibrosis (top, right) in ventricular septum endocardium of DMs mouse heart (DMs-Se). Right atrium stained with von Kossa stain (DMs-RA) displays calcification in this region. Masson Trichrome stain of left atrium (LA) and ventricle (LV) in DMs mice reveal fibrosis in these regions. Bar is equivalent to 150 μm.
FIG. 8(B) illustrates a Northern RNA blot showing: skActin, β-MHC, α-MHC, ANF RNA expression in representative WT and DMs left ventricles at 3 months of age. Gapdh RNA was assessed as a loading control.
FIG. 9 (or FIG. S5) graphically illustrates echocardiographic measurements of cardiac dimensions and function in WT (n=14) and DM (n=14) hearts at 6 wks of age. Abbreviations: IVSd: Interventricular septal wall thickness at end-diastole; LVPWd: Left ventricular (LV) posterior wall thickness at end-diastole; LVIDd: LV internal dimension at end-diastole; LVIDs: LV internal dimension at end-systole; FS (%): LV percent fraction shortening.
FIG. 10 (or FIG. S6) graphically illustrates Ca²⁺-contraction twitch dynamics in wild type (WT) and DM muscles at 25° C. Ca²⁺ transients and isometric twitch tension traces were recorded in WT (n=9) and DM (n=7) papillary muscles following steady-state pacing at 2 and 4 Hz and have been normalized to emphasize time course features. Values are means±SEM. Abbreviations: ΔRsyst—diast, change in Fura-2 fluorescence ratio from diastolic to peak systolic value; τdecay, time constant of Ca²⁺ transient decay; TP50-Ca, time from peak to 50% Ca²⁺ transient decay; TTP-T, time from stimulus to peak tension; TP50-T, time from peak to 50% tension decay. *p<0.05 vs. same group at 2 Hz. ̂p<0.05 vs. WT at the same pacing frequency (two-way ANOVA).
FIG. 11 (or FIG. S7) graphically illustrates twitch dynamics in WT and DM muscles at 37° C.
FIG. 11(A) graphically illustrates representative isometric twitch tension measured in right ventricular papillary muscles isolated from WT and DM papillary muscles. Traces were recorded following steady-state pacing at 5 Hz.
FIG. 11(B) graphically illustrates mean characteristics of twitch tension time courses in WT (n=7) and DM (n=6) papillary muscles. DM muscles exhibit significantly reduced time from peak to 50% relaxation and thus accelerated relaxation. Values are represented as mean±SEM. Abbreviations: TTP-T, time from stimulus to peak tension; TP50-T, time from peak to 50% tension decay. ** p<0.001 vs. WT.
FIG. 12 (or FIG. S8) graphically illustrates DM mutant mice sensitized to pressure overload following transverse aortic constriction (TAC).
FIG. 12(A) graphically illustrates Left ventricle (LV) to body weight (BW) ratios as well as in vivo echocardiographic assessment of cardiac size and function in 6 wk old WT and DM mutant mice, before (pre) and following (post) sham and TAC operation for 1 week. IVSd: Interventricular septal wall thickness at end-diastole; LVPWd: Left ventricular (LV) posterior wall thickness at end-diastole; FS (%): LV percent fraction shortening; LVIDd: LV internal dimension at end-diastole; LVIDs: LV internal dimension at end-systole. **p<0.01 vs. WT-sham, ##p<0.01 vs. DM-sham, ¶¶p<0.01 vs. WT-TAC. Trans-stenotic pressure gradients within WT (82.35±10 8 mmHg (n=7)) and DM (78.05±9.7 mmHg (n=7)) hearts were not significantly different. No significant changes in heart rates were observed between mice between groups.
FIG. 12(B) graphically illustrates cardiomyocyte length and widths plotted from WT (black line (n=3)) versus DM (red line (n=3)) mice pre and post-sham and TAC operation for 1 week. Red arrow highlights shift towards higher cardiomyocyte length in DM-TAC. Data is expressed as arbitrary units (A.U.). Left shift representative of increased cell width was only observed in cardiomyocytes isolated from WT-TAC. **p<0.01
FIG. 12(C) graphically illustrates ANF, β-MHC, α-MHC, sk-Actin, c-Actin and PLB RNA expression in left ventricles from mice pre and post-sham and TAC operation for 1 week (n=3 in each group); normalized to Gapdh RNA expression and expressed as a percentage (%) of WT-sham controls, which is set to 100%. *p<0.05, **p<0.01, ***p<0.001, n.s. represents not significant.
FIG. 13 (or FIG. S9) illustrates an exemplary schematic model of the mechanisms driving actin-myosin interactions in cardiac muscle, which are controlled by the effects of the myosin accessory protein, MLC2v and its phosphorylation status.
FIG. 13 (Top panel) illustrates MLC2v phosphorylation simultaneously increases the likelihood of myosin binding and force produced by each myosin binding event to heterogeneously increase myofilament calcium sensitivity and decrease rate of twitch relaxation across the heart wall in calcium-dependent myocardial contraction events involved in maintaining ventricular torsion and function.
FIG. 13 (Bottom panel) illustrates loss of these mechanisms results in less actin-myosin binding events and those that do bind produce less force by each myosin binding event, resulting in decreased myofilament calcium sensitivity. Loss in these events result in impaired twitch relaxation, resulting in loss of ventricular torsion, which leads to an adverse subendocardial workload, which then predisposes the heart to dilated cardiomyopathy and heart failure.
FIG. 14 illustrates data showing how MLC2v protein is detectable in blood serum of mice; and the detection of the phosphorylated form of MLC2v increases following myocardial infarction. Wild type C57BL/6 mice underwent surgically induced permanent left anterior descending branch ligation (myocardial infarction, MI) or SHAM operation (chest opened but no LAD ligation). Blood was collected from mice at 24 and 48 hours post-MI or SHAM operation (48 hours). Protein blot analysis of MLC2v expression in cardiac myofibrillar extracts (positive control) and blood serum of mice revealed that MLC2v could be detected as both non-phosphorylated (white arrow) and phosphorylated (black arrow, higher migration band which was confirmed via mass spec analysis) forms in blood serum of mice and that there is increased presence of the phosphorylated form in blood serum of mice at 48 hours post-MI (a time point coincident with cardiac troponin I detection (a current biomarker for myocardial damage, data not shown)). At 24 hours post-MI, MLC2v appears to be in the non-phosphorylated form (below). At this 24 hour time point, no cardiac troponin I could be detected in blood serum (data not shown), which suggests that the severity of damage may be significantly less at 24 hours.

### Materials and Methods

Generation of gene targeted mice. Mlc-2v genomic DNA was isolated from a 129-SV/J mouse genomic DNA library, as previously described (30). PCR-based mutagenesis was used to introduce (i) a single mutation (SM) of T to G in codon 15 of Mlc-2v as well as (ii) a double mutation (DM) from AG to GC in codon 14 and from T to G in codon 15 of Mlc-2v to generate targeted alleles for SM and DM mice, respectively. The SM changed codon 15 from Ser to Ala and simultaneously abolished a SstI site, whereas the DM changed codon 14 and 15 from Ser to Ala and also simultaneously abolished a SstI site. A pGKneo-tk cassette flanked by two loxP sites was inserted into intron 2 as a selectable marker in both targeted alleles such that it could subsequently be deleted by Cre mediated recombination. The targeting constructs were linearized with NotI before electroporation into R1 ES cells. G418-resistant ES clones were screened for homologous recombination by SstI digestion, followed by Southern blot analysis as previously described (31). To avoid the interference of the pGKneo-tk cassette with expression of the Ser15 to Ala15 and Ser14/15 to Ala14/15 alleles, the cassette was deleted in ES clones by transient transfection of the cre-encoding plasmid pmc-cre and selection with gancyclovir as described (32). Two independent homologous recombinant ES clones for each line were microinjected into C57BL/6J blastocysts and transferred into pseudopregnant recipients. SM and DM chimeric animals resulting from the microinjection were bred with C57BL/6J mice to generate germ line-transmitted agouti heterozygous SM and DM mice. PCR analysis was performed on tail DNA from mouse offspring from SM and DM intercrosses by using Mlc-2v primers (forward, CACTTGGTCATAGTCACTTGTG (SEQ ID NO:1); reverse, GGATGGATGCTATGCT GCCCAG (SEQ ID NO:1)) using standard procedures. Sequence analysis (Bio Applied Technologies Joint Inc., CA) was performed on PCR products to verify the presence of the mutations in SM and DM mice, using standard procedures. Both SM and DM offspring were backcrossed into the C57BL/6J background. Since SM and DM mice were backcrossed for at least ten generations into the C57BL/6J background, we utilized age-matched wild type C57BL/6J mice (Charles River Laboratories) as controls for all experiments. All animal procedures were in full compliance with the guidelines approved by UCSD Animal Care and Use Committee.

Two-Dimensional gel analysis. Multicellular myocardial preparations (600-900 mm×100-250 mm) were isolated and homogenized from mouse hearts as previously described (21). The homogenates were centrifuged at 120 g for 1 min, and the resulting pellet was washed with fresh relaxing solution and resuspended in relaxing solution containing 250 μg saponin/ml and 1% Triton X-100. After 30 min, the skinned preparations were washed with fresh relaxing solution and were dispersed in 50 ml relaxing solution in a glass Petri dish. The dish was kept on ice except during the selection of multiple preparations, which were subsequently placed in rehydration/sample buffer (Bio-Rad Laboratories). Myocardial homogenates were analyzed for non-phosphorylated and phosphorylated MLC-2v states using two-dimensional gel electrophoresis in a mini gel system (Bio-Rad Laboratories) as previously described (20). In brief, the first dimensional iso-electric focusing (IEF) tube gels containing 8 mM urea, 4% acrylamide-bisacrylamide (30% acrylamide/bisacrylamide solution; Bio-Rad Laboratories), 2% Triton X-100, 2% ampholyte (pH 4.1-5.9; Bio-Rad Laboratories), 0.02% ammonium persulfate, and 0.2% TEMED were prefocused first at 200 V for 15 min and then at 400 V for 15 min. The samples were then loaded onto the gels and electrofocused first at 500 V for 20 min and then at 750 V for 4 h 40 min. The IEF tube gels were ejected onto a 12.5% Tris-HCL Criterion Precast gel (Bio-Rad Laboratories) and electrophoresed at 150 V for 1 h 30 min. The gels were then silver stained at room temperature, as described by manufacturer's instructions. The percent MLC-2v phosphorylation was quantified by using densitometry.

Liquid Chromatography (LC)-Tandem Mass Spectrometery (MS/MS) analysis. Myofilament proteins were isolated from mouse hearts as previously described (33). Protein samples were separated by 12% sodium dodecyl sulfate (SDS)-polyacrylamide gel electrophoresis (PAGE) and visualized by Coomassie blue staining as previously described (33). The gel band corresponding to the MLC-2v protein (19 kDa) was excised and trypsinized as described by Shevchenko et al. (34). The extracted peptides were analyzed directly by LC-MS/MS using electrospray ionization. All nanospray ionization experiments were performed using a QSTAR-Elite™ hybrid mass spectrometer (ABSciex®) interfaced to a nanoscale reversed-phase high-pressure liquid chromatograph (Tempo™) using a 10 cm-180 micron ID glass capillary packed with 5-mm C18 Zorbax™ beads (Agilent®). The buffer compositions were as follows. Buffer A was composed of 98% H₂O, 2% ACN, 0.2% formic acid, and 0.005% TFA; buffer B was composed of 100% ACN, 0.2% formic acid, and 0.005% TFA. Peptides were eluted from the C-18 column into the mass spectrometer using a linear gradient of 5-60% Buffer B over 60 min at 400 ul/min. LC-MS/MS data was acquired in a data-dependent fashion by selecting the 4 most intense peaks with charge state of 2 to 4 that exceeds 20 counts, with exclusion of former target ions set to “360 seconds” and the mass tolerance for exclusion set to 100 ppm. Time-of-flight MS was acquired at m/z 400 to 1600 Da for 1 s with 12 time bins to sum. MS/MS data were acquired from m/z 50 to 2,000 Da by using “enhance all” and 24 time bins to sum, dynamic background subtract, automatic collision energy, and automatic MS/MS accumulation with the fragment intensity multiplier set to 6 and maximum accumulation set to 2 s before returning to the survey scan. Peptide identifications were made using paragon algorithm executed in Protein Pilot 2.0 (Life Technologies) with emphasis on biological modifications and phosphorylation in addition to Mascot™ (Matrix Sciences®). Peptides with confidence levels of above 95% were identified as positive.

Phosphorylation assays. Myofilament proteins were isolated from mouse hearts as previously described (33). MLC2v kinase reactions were performed at 30° C. using 50 pg of myofibrillar protein extract. For assessment of cardiac MLCK phosphorylation, reactions were performed using 1.7 nM of cardiac MLCK in 25 μl of kinase buffer (25 mM HEPES, pH7.6, 10 mM MgCl₂, 5 mM DTT, 20 mM NaCl, 0.2% triton, 2% glycerol, 0.5 mg/ml BSA and 0.5 mM [γ-³²P]-ATP at 267 cpm/pmol). For assessment of skeletal MLCK phosphorylation, reactions were performed using 2 nM of skeletal MLCK in 25 μl of kinase buffer supplemented with 1 mM Ca²⁺ and 1 mM calmodulin. Reactions were terminated after 15 minutes by the addition of SDS-sample buffer. Samples were separated by 15% SDS-PAGE gel, stained with Coomassie blue, and the level of MLC2v phosphorylation was visualized by autoradiography. The relative amount of phosphorylated and total MLC2v was determined by densitometric analyses. In addition, phosphorylated MLC2v proteins were excised from the gel and their radioactivity measured by liquid scintillation counting.

Ventricular Weight to Body Weight Ratios and Histological analysis. Mice were anesthetized with ketamine/xylazine and weighed to determine total body weight. Hearts were then removed, including all major vessels, connective tissue and atria were dissected away. The left ventricles were separated, blotted and weighed. Paraffin-embedded cardiac sections (8 mm thick) were stained with hematoxylin and eosin and Masson Trichrome stain as previously described (35). A von Kossa (Sigma Aldrich) staining assay was also performed on paraffin embedded cardiac sections according to the manufacturer's instructions.

Echocardiography. Mice were anesthetized with 1% isoflurane and subjected to echocardiography as previously described (36).

Morphometric analyses of isolated adult mouse cardiac myocytes. Adult cardiac myocytes were isolated from mouse hearts as previously described (35). Cell length and width measurements were performed on isolated adult cardiac myocytes using the National Institutes of Health (NIH) Image J software.

Electron microscopy. Hearts were first perfused with a high potassium phosphate buffered saline solution containing 77 mM NaCl, 4.3 mM Na₂HPO₄.7H₂O, 1.47 mM KH₂PO₄ and 62.7 mM KCl, followed by perfusion with 2% paraformaldehyde in 0.1 M sodium cacodylate buffer, pH 7.4. The left ventricle free wall was subsequently cut into 1 mm pieces and immersed in a modified Karnovsky's fixative (1.5% glutaraldehyde, 3% paraformaldehyde and 5% sucrose in 0.1 M sodium cacodylate buffer, pH 7.4) for at least 8 hours, postfixed in 1% osmium tetroxide in 0.1 M cacodylate buffer for 1 hour and stained en bloc in 1% uranyl acetate for 1 hour. Hearts were dehydrated in ethanol, embedded in epoxy resin, sectioned at 60 to 70 nm, and picked up on Formvar and carbon-coated copper grids. Grids were stained with uranyl acetate and lead nitrate, viewed using a JEOL 1200EX II (JEOL, Peabody, Mass.) transmission electron microscope and photographed using a Gatan digital camera (Gatan, Pleasanton, Calif.).

RNA analysis. Total RNA was extracted from left ventricles using TRIzol (Invitrogen). Dot blot analysis was performed as previously described (35).

Measurement of Ca²⁺-mediated force dynamics in isolated intact papillary muscles. Right ventricular papillary muscles were isolated from mouse hearts, mounted and calibrated in a cardiac tissue culture chamber as previously described (35). After setting the muscle at optimal length, the top and side aspects of the muscle were photographed and digitized to determine muscle cross sectional area. Subsequently pacing was increased to 2 Hz, and the muscle was allowed to equilibrate for an additional 20-30 minutes. Ca²⁺ transients and twitch tension were measured simultaneously in isolated right ventricular papillary muscles at 25° C. For these measurements, the perfusion was stopped and the bathing solution replaced with a loading solution containing the membrane-permeable fluorescent Ca²⁺ indicator Fura-2AM (2 μM final concentration, Invitrogen, Inc.). The muscle was allowed to load at room temperature for 25-30 minutes, after which the bath temperature was set to its corresponding value (25° C.) and the perfusion and pacing were resumed. Muscles were imaged using an extra-long working distance 20× objective. Ratiometric measurement of Fura-2 fluorescence was accomplished by illuminating the muscle with rapidly alternating (333 Hz) 340/380 nm light. Excitation wavelength switching was performed using a fast filter switcher (Lambda DG-4, Sutter Instrument, Inc.). Fura-2 emission (wavelength 540 nm) was then filtered and measured with a photomultiplier tube system (PMT-100, Applied Scientific Instrumentation) and processed by a Data Acquisition Processor (5216a, Microstar Laboratories, Inc.) running custom programs. Experimental protocols, including patterns of pacing and length perturbations, were designed and run using custom software running on the host PC.

Myofilament Ca²⁺ activation computational model with three-state cross bridge cycle. A recently published two-state actin-myosin crossbridge cycling computational model of myofilament Ca²⁺ activation (17) was modified to a 3-state model (37) to gain insight into the molecular actions of MLC2v phosphorylation. This modification consisted of a detached crossbridge state and two attached states, pre-power stroke and post-power stroke (C, Mₚᵣ, and Mₚₒ respectively, FIG. 1c). Simulated relative contractile force was computed as the fraction of crossbridges in the post-power stroke state. The original features of the computational model showed that Ca²⁺ activation events were more potent than crossbridge binding events in producing cooperative activation of the nearest-neighbor interactions between overlapping tropomyosin molecules along the actin filament as well as other physiological behavior. The new version of the model makes the simplification that only Ca²⁺ activation events are communicated among nearest neighbors, a property described by the coefficient γ (Table S1). The behavior of several interacting myosin binding sites is represented using a Markov model (17). Whereas each binding site was previously assumed to reside in one of three states (blocked, closed, or open), the above simplification allows closed and open states to be merged and the number of total Markov model states is reduced substantially. All simulations reported here were performed assuming 13 interacting myosin binding sites with periodic boundary conditions (38). Merging closed and open states necessitated definition of a new conditional probability, μ, which describes the fraction of binding sites in this state that are capable of transitioning to a blocked state. The quantity μ is analogous to the conditional probability φ given previously (17) and is defined by:

$$μ = \frac{P \{ C \}}{P \{ C M \}} where P \{ C \}$$

is the probability associated with the C state and P {CM} is the global probability associated with the lumped closed/open state as described by the Markov model states. Model parameters and their values are listed in Table S1. Rates of Ca²⁺ binding and dissociation from troponin C (k_{Ca}⁺ and k_{Ca}⁻), as well as rates governing tropomyosin shifting between blocked and closed positions (k_{B}⁺ and k_{B}⁻) were based on values determined previously (17), with only minor modifications to account for general species-specific differences. The three-state crossbridge model (37) introduced five new model parameters, including f (crossbridge attachment rate) g (detachment rate of pre-power stroke crossbridges), h_{f} (forward power stroke rate), h_{b} (reverse power stroke rate), and g_{xb} (detachment of post-power stroke crossbridges). Parameter values were coarsely adjusted to produce a crossbridge duty cycle (average fraction of cycle time spent bound to actin) of ˜20%, in accordance with previous modeling work (17). All simulations assumed constant sarcomere length, meaning that force is produced in proportion to the occupancy of the Mₚₒ state. Force produced by the model was calculated as the product of individual crossbridge stiffness (k_{xb}), crossbridge distortion induced by the power stroke (x₀), and the number of attached, post-powerstroke myosin heads P {Mₚₒ}:

F=k_{xb}xₒP{Mₚₒ}

The value of k_{xb} was set to 125 kPa/nm in order to match mean peak twitch tension at 4 Hz pacing frequency and 25° C. bath temperature. Five model parameters, f, h_{f}, h_{b}, g_{xb}, and x₀, were identified as potentially dependent on MLC2v phosphorylation. Crossbridge attachment rate (f) was assumed to increase with MLC2v phosphorylation due to increased diffusion of the myosin head away from the thick filament backbone (Mechanism 1, FIG. 3A). Mechanism 2 (FIG. 3B), in which phosphorylation increases stiffness of the myosin lever arm domain, was assumed to increase crossbridge strain induced by the power stroke (x₀), which would in turn slow the power stroke rate (decrease h_{f} and increase h_{b}) and increase rate of detachment of post-power stroke crossbridges (g_{xb}). In order to conveniently summarize MLC2v phosphorylation-based changes to these parameters, the final value of each was re-calculated in the following generic format:

k_{final}=k_{base}(1+pₖQ_{MLC2v-P})

where k generically represents one of the five model parameters, k_{base} is that parameter's baseline (non-phosphorylated) value (Table S1), pₖ is the corresponding MLC2v-P weighting coefficient, and Q_{MLC2v-P} is the fractional MLC2v phosphorylation level. Thus, values of the five weighting coefficients describe the sensitivity of corresponding parameters to Q_{MLC2v-P}. MLC2v-P weighting coefficients were determined by comparing simulation output with experimental measurements in skinned mouse myocardium at 15° C. reported by Stelzer et al. (21). Essential characteristics of the published data (20,21) include the observations that MLC2v phosphorylation (i) increased maximum Ca²⁺-activated force by 40%, (ii) increased Ca²⁺ sensitivity of force, and (iii) did not significantly change the rate of force redevelopment following slack/re-stretch of the muscle (kₜᵣ), even when several levels of Ca²⁺ activation were tested. Parameters of the myofilament model were first adjusted to fit the force-Ca²⁺ relation observed in the absence of MLC2v phosphorylation (Table S1). Leaving all other model parameters unchanged, QMLC2v-P was set to 0.39 and weighting coefficients representing Mechanisms 1 and 2 were adjusted until responses observed at 39% MLC2v phosphorylation were matched (main text, FIG. 4D). Table S2 lists the resulting weighting coefficients.

Muscle twitch dynamic simulations using new computational model. The new computation model parameters (FIG. 4C) were first coarsely adjusted to reflect increased steady-state Ca²⁺ sensitivity and cooperativity observed in intact cardiac muscle (20), and twitch forces were simulated by driving the model with a representative Ca²⁺ transient recorded in WT preparation paced at 4 Hz and 25° C. Parameters were then fine-tuned such that a simulated twitch matched a representative record from a DM papillary muscle at the same temperature and pacing rate. A WT twitch was then predicted simply by increasing Q_{MLC2v-P} from 0 to 0.31 (the value measured in WT mouse myocardium), which altered crossbridge kinetics according to the sensitivities determined earlier (Table S2).

Urea Glycerol Gel Analysis Method to Quantify MLC2v-P. Urea glycerol gel methods were done essentially as previously described (22, 39). Briefly, mouse hearts (3 months old) were rapidly excised, arrested [35 mM KCl, 100 mM NaCl, 0.36 mM NaH2PO4, 1.75 mM CaCl2, 1.08 mM MgCl2, 21 mM NaHCO3, 5 mM glucose, 5 U/L insulin and 0.08 g/L BSA] and mounted on a Lagendorff perfusion system utilizing 90 mmHg constant pressure perfusion at 37° C. A small custom plastic balloon was inserted into the left ventricle (LV) chamber through the mitral orifice. Hearts were perfused with an oxygenated Tyrode solution [7.4 mM KCl, 127 mM NaCl, 0.36 mM NaH2PO4, 1.75 mM CaCl₂, 1.08 mM MgCl2, 21 mM NaHCO3, 5 mM glucose, 5 U/L insulin and 0.08 g/L BSA.] and paced at 250 bpm. Hearts were allowed to equilibrate and stabilize with 5-10 mmHg preload. A Frank-Starling protocol was utilized to determine the appropriate volume for 0 mmHg preload. Upon cessation of contractions, pacing was turned off and volume was changed to the appropriate preload in hearts for 30 minutes and then immediately flash frozen in liquid N₂. Endocardial and epicardial segment sections were performed on frozen hearts in 60% glycerinating solution in relaxing solution including 84 mM leupeptin, 20 mM, E-64 and 80 mM PMSF. Approximately 20 mg of frozen tissue was pulverized to a fine powder and solubilized in 50% glycerol containing 84 mM leupeptin, 20 mM E-64, and 80 mM PMSF and 620 ml of freshly prepared urea sample buffer (9 M urea, 50 mM Tris pH 8.6, 300 mM glycine, 5 mM DTT, and 0.001% bromophenol blue). The proteins were separated by urea glycerol PAGE. Myosin regulatory light chain 2 ventricular (MLC2v) and the MLC2v phosphorylated (MLC2vP) bands were identified by western blot and Pro-Q Diamond stain. Specific mouse MLC2v monoclonal antibodies (1:1000) that recognizes human and rat ventricular MLC2v (amino acids 45-59) (Enzo Life Sciences, Ab manufactured by BioCytex) were used for western blot analysis. To determine the relative phosphorylation level of MLC2v in epi- and endocardial tissues, the gels were stained with Coomassie blue according to manufacturer's instructions. The densitometry analysis of the protein bands was carried out with 1Dscan EX (Scanalytics Inc., Rockville, Md., USA) software. A range of loadings was used per sample and the integrated OD for each loading was determined. The linear range of the OD-loading relation was determined and the slope (m) of this relation was calculated by using linear regression analysis. The slope of the MLC2v and the MLC2vP was used to obtain the percentage of MLC2vP according to:

MLC2vP(%)=(mMLC2vP×100)/(mMLC2vP+mMLC2v).

Computational model of LV torsion. A finite element model of the mouse left ventricle (LV) was generated. LV geometry was approximated as a thick-walled, truncated ellipse of revolution whose dimensions (wall thickness, focal length, and end-diastolic volume) were based on MR-derived anatomical data obtained as a part of this study. A transmural pattern of myofiber orientation was assumed based on published gradients in the murine LV free wall (40). A three-element Windkessel model of the circulation was used to provide appropriate ventricular afterload. Mouse-specific parameter values for the circulatory model were taken from published in vivo measurements (41). A five millisecond-delay in activation was assumed across the thickness of the LV wall, based on typical conduction velocities in mouse myocardium (42). Ca²⁺ and length-dependent myocardial contractile force was simulated using the model of Rice et al. (37). A modification of the original parameter values was used for this model to reproduce responses reported for intact mouse myocardium including pCa₅₀ and Hill coefficient of steady-state force activation (42). Parameters describing myosin crossbridge kinetics were modified to vary as a function of phosphorylated MLC2v in accordance with data showing that phosphorylation increases crossbridge binding and accumulation of crossbridges in the strongly-bound state (20,21). A single cardiac beat was simulated by applying a realistic Ca²⁺ transient (43), regionally adjusted for activation delay, to each point throughout the mesh. The time course of myocardial deformation was obtained through solution of model equations under this time-varying input. Ventricular torsion was calculated using the same method as described for the MR tagging measurements. Two separate simulations were performed. In the first, MLC2v phosphorylation was assumed to vary linearly across the thickness of the LV wall, with 30% phosphorylation at the endocardium and 45% at the epicardium. This transmural difference of 15% phosphorylated MLC2v is based on measurements made in isolated rat and mouse ventricle (FIG. 4A) (22). In the second simulation, phosphorylation was assumed to be zero at all locations in the ventricle to mimic conditions in the mutant mouse. The density of myofiber stroke work (SWD) was calculated for each point in the finite element mesh as the area under the curve formed by plotting contractile tension (σ(t)) as a function of Lagrangian fiber strain (ε(t)) during the ejection phase of the cardiac cycle:

$$S W D = {\int }_{{t}_{ED}}^{{t}_{EE}} σ ( t ) d ɛ ( t )$$

Here, t_{ED} and t_{EE} refer to the time at end diastole and end ejection, respectively.

Magnetic Resonance Imaging (MRI) and left ventricle (LV) torsion analysis. In vivo murine cardiac imaging was performed on a 7T horizontal-bore MR scanner (Varian magnet with a Buker console), equipped with a 21 cm bore. Mice were anesthetized with isoflurane and imaged in a 2.5 cm Bruker volume coil. Body temperature and the electrocardiogram were monitored. Heart rate was maintained around 400 bpm. Cine anatomical imaging was performed using the Fast Low Angle Shot sequence (FLASH) with flip angle=15°, echo time=2.8 ms, repetition time=6 ms, data matrix=128×128, field of view=2.0 cm, slice thickness=1 mm, and 4 averages. Myocardial tagging was performed using spatial modulation of magnetization (SPAMM) (44). The tag thickness was 0.3 mm and tag line separation was 0.7 mm, which allowed for 2-3 taglines to be placed across the ventricular wall of the mouse heart. Parameters for the image acquisition were the same as the cine acquisition except for the inclusion of the tagging module and the number of averages was increased to 20. During imaging, the long axis of the left ventricle was first identified. Both cine and SPAMM tagged short axis images were then taken at the base and apex of the left ventricle. Six-week old wild type and mutant mice were scanned (n=3 for each group). For one mouse, the total imaging time was approximately 30 minutes. Tag intersections near the epicardium of the LV free wall are tracked from end diastole to end systole in the basal and apical slice. Torsion was calculated as the circumferential angular displacement between two points, one near the base and one near the apex, normalized by the vertical distance separating them (45). LV volumes were estimated from long axis MR images using Simpson's disc summation method:

$$V = {\sum }_{i = 1}^{n} \frac{π t}{4} {D}_{i}^{2}$$

This method approximates the LV cavity by a stack of n discs, each having their own diameter Dᵢ. The thickness of each disk, t, is the distance corresponding to a single image pixel. Disc diameters are taken as the horizontal distance between endocardial boundary points along a single line of pixels in the long axis MR image. Estimated end-diastolic volumes (EDV) and end-systolic volumes (ESV) were then used to calculate % ejection fraction (EF) according to the formula:

$$E F = \frac{E D V - E S V}{E D V} \times 100 \%$$

In vivo pressure overload model. Mice (6 wks old) were anesthetized with ketamine/xylazine, and transverse aortic constriction (TAC) was performed as previously described (46). At 7 days following surgery, the pressure gradients generated by aortic banding were measured by introducing high-fidelity pressure transducers into the left and right common carotids.

Statistical Analysis. Data presented in the text and figures are expressed as mean values±standard error of the mean. Significance was evaluated by the two-tailed student's t-test or repeated measures ANOVA. p<0.05 was considered statistically significant.

### REFERENCES

1. J. A. Spudich, The myosin swinging cross-bridge model. Nat. Rev. Mol. Cell. Biol. 2, 387 (2001).
2. H. Morita, J. Seidman, C. E. Seidman, Genetic causes of human heart failure. J. Clin. Invest. 115, 518 (2005).
3. A. M. Gordon, E. Homsher, M. Regnier, Regulation of contraction in striated muscle. Physiol. Rev. 80, 853 (2000).
4. H. L. Sweeney, B. F. Bowman, J. T. Stull, Myosin light chain phosphorylation in vertebrate striated muscle: regulation and function. Am. J. Physiol. 264, 1085 (1993).
5. A. C. Hinken, R. J. Solaro, A dominant role of cardiac molecular motors in the intrinsic regulation of ventricular ejection and relaxation. Physiology (Bethesda) 22, 73 (2007).
6. J. S. Davis et al., The overall pattern of cardiac contraction depends on a spatial gradient of myosin regulatory light chain phosphorylation. Cell 107, 631 (2001).
7. J. van Der Velden et al., Effects of calcium, inorganic phosphate, and pH on isometric force in single skinned cardiomyocytes from donor and failing human hearts. Circulation 104, 1140 (2001).
8. J. van Der Velden et al., Increased Ca2+ sensitivity of the contractile apparatus in end-stage human heart failure results from altered phosphorylation of contractile proteins. Cardiovasc. Res. 57, 37 (2003).
9. I. Morano, Effects of different expression and posttranslational modifications of myosin light chains on contractility of skinned human cardiac fibers. Basic Res. Cardiol. 87 Suppl 1, 129 (1992).
10. D. Szczesna et al., Familial hypertrophic cardiomyopathy mutations in the regulatory light chains of myosin affect their structure, Ca2+ binding, and phosphorylation. J. Biol. Chem. 276, 7086 (2001).
11. K. Poetter et al., Mutations in either the essential or regulatory light chains of myosin are associated with a rare myopathy in human heart and skeletal muscle. Nat. Genet. 13, 63 (1996).
12. S. B. Scruggs et al., A Novel, In-Solution Separation of Endogenous Cardiac Sarcomeric Proteins and Identification of Distinct Charged Variants of Regulatory Light Chain. Mol. Cell. Proteomics. 9, 1804 (2010).
13. B. P. Herring, P. J. Gallagher, J. T. Stull, Substrate specificity of myosin light chain kinases. J. Biol. Chem. 267, 25945 (1992).
14. M. H. Dickinson et al., Phosphorylation-dependent power output of transgenic flies: an integrated study. Biophys. J. 73, 3122 (1997).
15. A. Sanbe et al., Abnormal cardiac structure and function in mice expressing non-phosphorylatable cardiac regulatory myosin light chain 2. J. Biol. Chem. 274, 21085 (1999).
16. M. Ikebe, D. J. Hartshorne, M. Elzing a, Identification, phosphorylation, and dephosphorylation of a second site for myosin light chain kinase on the 20,000-dalton light chain of smooth muscle myosin. J. Biol. Chem. 261, 36 (1986).
17. S. G. Campbell et al., Coupling of Adjacent Tropomyosins Enhances Crossbridge Mediated Cooperative Activation in a Markov Model of the Cardiac Thin filament. Biophys. J. 98, 2254 (2010).
18. R. J. Levine et al., Myosin light chain phosphorylation affects the structure of rabbit skeletal muscle thick filaments. Biophys. J. 71, 898 (1996).
19. M. J. Greenberg et al., The molecular effects of skeletal myosin regulatory light chain phosphorylation. Am. J. Physiol. Regul. Integr. Comp. Physiol. 297, R265 (2009).
20. M. Olsson, J. Patel, D. Fitzsimons, J. Walker, R. L. Moss, Basal myosin light chain phosphorylation is a determinant of Ca2+ sensitivity of force and activation dependence of the kinetics of myocardial force development. Am. J. Physiol. Heart Circ. Physiol. 287, H2712 (2004).
21. J. E. Stelzer, J. R. Patel, R. L. Moss, Acceleration of stretch activation in murine myocardium due to phosphorylation of myosin regulatory light chain. J. Gen. Physiol. 128, 261 (2006).
22. C. Hidalgo et al., Effect of diastolic pressure on MLC2v phosphorylation in the rat left ventricle. Arch. Biochem. Biophys. 456, 216 (2006).
23. M. W. Ashford Jr. et al., Occult cardiac contractile dysfunction in dystrophin-deficient children revealed by cardiac magnetic resonance strain imaging. Circulation 112, 2462 (2005).
24. Y. T. Tan et al., The pathophysiology of heart failure with normal ejection fraction: exercise echocardiography reveals complex abnormalities of both systolic and diastolic ventricular function involving torsion, untwist, and longitudinal motion. J. Am. Coll. Cardiol. 54, 36 (2009).
25. R. M. Sester et al., Persistent abnormal left ventricular systolic torsion in dilated cardiomyopathy after partial left ventriculectomy. J. Thorac. Cardiovasc. Surg. 126, 48 (2003).
26. R. E. Henson et al., Left ventricular torsion is equal in mice and humans. Am. J. Physiol. Heart Circ. Physiol. 278, H1117 (2000).
27. S. J. D. Brecker. The importance of long axis ventricular function. Heart 84, 577 (2000).
28. C. L. Mummery, R. P. Davis, J. E. Krieger, Challenges in using stem cells for cardiac repair. Sci Transl Med. 2, 27 ps17 (2010).
29. [blank]
30. J. Chen et al., Selective requirement of myosin light chain 2v in embryonic heart function. J. Biol. Chem. 273, 1252 (1998).
31. Q. Zhou et al., Ablation of Cypher, a PDZ-LIM domain Z-line protein, causes a severe form of congenital myopathy. J. Cell Biol. 155, 605 (2001).
32. J. Chen, S. W. Kubalak, K. R. Chien, Ventricular muscle-restricted targeting of the RXRalpha gene reveals a non-cell-autonomous requirement in cardiac chamber morphogenesis. Development 125, 1943 (1998).
33. C. W. Tong, R. D. Gaffin, D. C. Zawieja, M. Muthuchamy, Roles of phosphorylation of myosin binding protein-C and troponin I in mouse cardiac muscle twitch dynamics. J. Physiol. (Lond) 558, 927 (2004).
34. A. Shevchenko, M. Wilm, O. Vorm, M. Mann, Mass spectrometric sequencing of proteins silver-stained polyacrylamide gels. Anal. Chem. 68, 850 (1996).
35. F. Sheikh et al., An FHL1-containing complex within the cardiomyocyte sarcomere mediates hypertrophic biomechanical stress responses in mice. J Clin Invest 118, 3870 (2008).
36. N. Tanaka et al., Transthoracic echocardiography in models of cardiac disease in the mouse. Circulation 94, 1109 (1996).
37. J. J. Rice, F. Wang, D. M. Bers, P. P. de Tombe, Approximate model of cooperative activation and crossbridge cycling in cardiac muscle using ordinary differential equations. Biophys. J. 95, 2368 (2008).
38. P. Vibert, R. Craig, W. Lehman, Steric-model for activation of muscle thin filaments. J. Mol. Biol. 266, 8 (1997).
39. H. L. Granzier et al., Truncation of titin's elastic PEVK region leads to cardiomyopathy with diastolic dysfunction. Circ. Res. 105, 557 (2009).
40. G. J. Strijkers et al., Diffusion tensor imaging of left ventricular remodeling in response to myocardial infarction in the mouse. NMR Biomed. 22, 182 (2009).
41. P. Segers et al., Conductance catheter-based assessment of arterial input impedance, arterial function, and ventricular-vascular interaction in mice. Am. J. Physiol. Heart Circ. Physiol. 288, H1157 (2005).
42. G. E. Morley et al., Characterization of conduction in the ventricles of normal and heterozygous Cx43 knockout mice using optical mapping. J. Cardiovasc. Electrophysiol. 10, 1369 (1999).
43. W. D. Gao, N. G. Perez, E. Marban, Calcium cycling and contractile activation in intact mouse cardiac muscle. J. Physiol. (Lond) 507, 175 (1998).
44. L. Axel, L. Dougherty, M R imaging of motion with spatial modulation of magnetization. Radiology 171, 841 (1989).
45. J. Zhong, W. Liu, X. Yu, Characterization of three-dimensional myocardial deformation in the mouse heart: an MR tagging study. J. Magn. Reson. Imaging 27, 1263 (2008).
46. H. Rockman et al., Segregation of atrial-specific and inducible expression of an atrial natriuretic factor transgene in an in vivo murine model of cardiac hypertrophy. Proc. Natl. Acad. Sci. U.S.A. 88, 8277 (1991).

Supplementary Tables:

TABLE S1
Myofilament model parameter sets. Parameters were obtained
by fitting responses to measurements obtained in skinned mouse
myocardial preparations⁴ and in right ventricular papillary
muscles isolated from DM MLC2v mutant mice. Myosin-related
parameters (f, g, h_{f}, h_{b}, g_{xb}, and x₀) describe the activity
of myosin in the absence of MLC2v phosphorylation.

| | Values Fitted | Values Fitted to | |
|---|---|---|---|
| | to Data from Skinned | Data from | |
| Parameter | Mouse Myocardium, | Intact DM Mouse | |
| Name | 15° C. | Papillary Muscle, 25° C. | Units |
| k_{Ca}⁺ | 0.09 | 0.09 | μM ms⁻¹ |
| k_{Ca}⁻ | 0.47 | 0.625 | ms⁻¹ |
| k_{B}⁺ | 50 | 350 | ms⁻¹ |
| k_{B}⁻ | 0.327 | 0.327 | ms⁻¹ |
| γ_{B} | 50 | 300 | — |
| q | 0.5 | 0.5 | — |
| f | 5.10E−03 | 2.22E−02 | ms⁻¹ |
| g | 1.80E−03 | 7.80E−03 | ms⁻¹ |
| h_{f} | 5.12E−02 | 0.22 | ms⁻¹ |
| h_{b} | 1.02E−02 | 4.44E−02 | ms⁻¹ |
| g_{xb} | 5.12E−02 | 0.22 | ms⁻¹ |
| x₀ | 7 | 7 | nm |

TABLE S2
Coefficients describing the sensitivity of myofilament model
parameters to MLC2v phosphorylation. Values were determined
by fitting model responses to data obtained in skinned mouse
myocardial preparations⁴ at two different levels of
phosphorylated MLC2v.

| | | Name | Value |
|---|---|---|---|
| 1 | Mechanism | p_{f} | 2 |
| 2 | Mechanism | p_{hf} | −1 |
| | | p_{hb} | 1 |
| | | p_{gxb} | 0.2 |
| | | pₓ₀ | 0.2 |

A number of embodiments of the invention have been described. Nevertheless, it will be understood that various modifications may be made without departing from the spirit and scope of the invention. Accordingly, other embodiments are within the scope of the following claims.

1. A method for predicting or diagnosing a heart disease or a defect in cardiac muscle contractility in an individual, or a defect in rate of cardiac muscle twitch relaxation and/or ventricular torsion in an individual or in a cardiac cell, or detecting a cardiac trauma, comprising
(a) determining the presence or absence of, or the extent of, myosin light chain-2 (MLC2v) protein phosphorylation in a cardiac cell, an extracellular fluid, a serum or a blood serum or blood sample,
wherein a hypo-phosphorylated MLC2v protein, or non-phosphorylated MLC2v protein in a cardiac cell, and/or release of a phosphorylated MLC2v form into an extracellular fluid, a serum or a blood serum or blood sample, is predictive or diagnostic of a heart disease or a defect in cardiac muscle contractility, or a defect in rate of cardiac muscle twitch relaxation and/or ventricular torsion, or detects a cardiac trauma;
(b) the method of (a), wherein the MLC2v protein is phosphorylated, hypo-phosphorylated or non-phosphorylated in one or more or all serine residues in the MLC2v protein;
(c) the method of (b), wherein the individual, cardiac cell, extracellular fluid, serum or blood serum or sample, is murine or human (murine or human derived);
(d) the method of (c), wherein serine residue 14, or serine residue 15, or serine residue 14 and serine residue 15, or non-human or non-murine equivalent serine residues thereof, are not phosphorylated or phosphorylated;
(e) the method of (a), wherein the MLC2v protein is mutated or is a mutant protein, or is a non-wild type protein, in that one or more wild type serine residue or residues is missing or changed to a non-serine amino acid residue or residues;
(f) the method of (e), wherein the MLC2v protein is a mutant protein or non-wild type such that serine residue 14, or serine residue 15, or serine residue 14 and serine residue 15, or non-human or non-murine equivalent serine residues thereof, are is missing or changed to a non-serine amino acid residue or residues;
(g) the method of (e) or (f), wherein determining whether a MLC2v protein is mutated or is a mutant protein, or is a non-wild type protein, is by a method comprising sequencing (all or the relevant part of) the individual's or the cell's genome or transcriptome or MLC2v protein transcript;
(h) the method of any of (a) to (g), wherein the state of hypo-phosphorylation or non-phosphorylation in one or more or all normally or wild type phosphorylated residues in the MLC2v protein is measured or determined in vitro, ex vivo or in vivo; or
(i) the method of (a), wherein the presence or absence of, or the extent of, myosin light chain-2 (MLC2v) protein phosphorylation in the individual, cardiac cell, extracellular fluid, serum or blood serum or sample, is determined by a method comprising use of an antibody or a monoclonal or a polyclonal specific for a phosphorylated serine, or a serine MLC2v S14/S15 phosphorylation site; or comprising use of positron emission tomography (PET), computed tomography (CT), magnetic resonance imaging (MRI), nuclear magnetic resonance imaging (NMRI), or magnetic resonance tomography (MRT) and/or TOI.

2. A method for screening for a composition that can treat, ameliorate, prevent or reverse a heart disease or a congestive heart failure in an individual, or a defect in cardiac muscle contractility, or a defect in rate of cardiac muscle twitch relaxation and/or ventricular torsion in an individual or a cardiac muscle cell, comprising
(1) (a) providing a composition and a cardiac muscle cell, or a cultured cardiac cell, or a cardiac cell extract, or an equivalent cell or extract, or a serum or blood serum or sample;
(b) administering the composition to the cardiac muscle cell, cultured cardiac cell, cardiac cell extract, or equivalent cell or extract, or serum or blood serum or sample; and
(c) measuring or detecting an increase in the relative state of phosphorylation of MLC2v protein in the cardiac muscle cell, cultured cardiac cell, cardiac cell extract, or equivalent cell or extract, or serum or blood serum or sample,
wherein identifying a composition that can increase the relative state of phosphorylation of MLC2v protein in the cardiac muscle cell, cultured cardiac cell, cardiac cell extract, or equivalent cell or extract, or serum or blood serum or sample, identifies a composition that can treat, ameliorate, prevent or reverse a heart disease or a congestive heart failure in an individual, or a defect in cardiac muscle contractility, or a defect in rate of cardiac muscle twitch relaxation and/or ventricular torsion in an individual or a cardiac muscle cell;
(2) the method of (1), wherein the composition comprises a peptide or a protein, a small molecule, a nucleic acid, a carbohydrate or a polysaccharide or a lipid;
(3) the method of (1) or (2), wherein the composition is formulated for administration intravenously (IV), parenterally, orally, or by liposome or vessel-targeted nanoparticle delivery, or the composition comprises a pharmaceutical composition administered in vivo;
(4) the method of any of (1) to (3), wherein the composition increases the activity of or activates a kinase, or a myosin light chain kinase (MLCK); or
(5) the method of (1), wherein the presence or absence of, or the extent of, myosin light chain-2 (MLC2v) protein phosphorylation in a cardiac cell, or serum or blood serum or sample, is determined by a method comprising use of an antibody (monoclonal or polyclonal) specific for a phosphorylated serine, or a serine MLC2v S14/S15 phosphorylation site; or comprising use of positron emission tomography (PET), computed tomography (CT), magnetic resonance imaging (MRI), nuclear magnetic resonance imaging (NMRI), or magnetic resonance tomography (MRT) and/or TOI.
